(19) United States
(12) Patent Application Publication
(10) Pub. No.: US 2003/0143745 A1
(43) Pub. Date: Jul. 31, 2003

(54) Genetically modified bacterial strains and novel vectors for use in expressing and assaying natural products

(76) Inventors: Asuncion Martinez, Somerville, MA (US); Steve Kolvek, Somerville, MA (US)

Correspondence Address: ROSS J. OEHLER, AVENTIS PHARMACEUTICALS INC., ROUTE 202-206, MAIL CODE: D303A, BRIDGEWATER, NJ, 08807, US

(21) Appl. No.: 10/295,362

(22) Filed: Nov. 15, 2002

Related U.S. Application Data
(60) Provisional application No. 60/335,181, filed on Nov. 15, 2001.

(30) Foreign Application Priority Data
Jun. 13, 2002 (GB) 0213617.4

(51) Int. Cl. C12N 1/21; C12N 15/74

(52) U.S. Cl. 435/486; 435/252.3; 435/320.1

(57) ABSTRACT

Provided herein are heretofore-unknown strains of Streptomyces that are unable to express undecylprodrodigiosin, actinorhodin, or both, as well as novel vectors that permit the creation of chromosomal mutations in Streptomyces and do not insert selection markers in the genome of Streptomyces, along with vectors and methods for transferring large segments of DNA into the Streptomyces and Psuedomonas chromosome by conjugation.

### FIELD OF THE INVENTION

The present invention relates to novel and heretofore-unknown strains of Streptomyces that are unable to express undecylprodrodigiosin, actinorhodin, or both. Also provided are novel vectors that permit the creation of chromosomal mutations in bacteria, particularly Streptomyces and Pseudomonas, that do not insert selection markers into the bacterial genome, along with vectors and methods for transferring large segments of DNA into the bacterial chromosome by conjugation.

### BACKGROUND OF THE INVENTION

Natural products have been a tremendously rich source of pharmaceutical molecules, accounting for greater than 30% of all human therapeutics and more than 60% of anti-infective and anti-cancer drugs. Despite the advances in high throughput screening technology and attempts to isolate and culture microorganisms from exotic environments, the rate of discovery of novel products has declined. However, it is clear that the vast majority of microorganisms in the environment are still unknown, and that most of them are unculturable under standard laboratory conditions. Since the number of microbial species in the soil that cannot be grown in the laboratory represents at least 98% of the total population, these uncultured species might provide a large, untapped pool of novel natural products. Modern molecular biology offers a way to circumvent this problem: DNA of unculturalable organisms can be isolated directly from environmental samples, cloned into suitable vectors, and expressed in a surrogate host that can be grown in the laboratory. Unfortunately, heretofore known molecular biology methods have limited applications in characterizing and examining natural products because they generally do not permit researchers to transfer large pieces of DNA, i.e., up to 100 kilobases or more, from one species to another. Thus, the benefits of assaying for natural products, including high throughput screening methods, have not been maximized.

Streptomyces lividans is a well-characterized organism that readily has applications in identifying and characterizing natural products produced from recombinant DNA technology. Unfortunately, however, S. lividans produces two pigmented antibiotics, undecylprodrodigiosin and actinorhodin, that can interfere with various assay methods, particularly antibacterial assays as well as those that utilize a portion of the electromagnetic spectrum, e.g., fluorescence, phosphorescence, infrared, ultraviolet, etc. Consequently, the application of S. lividans in such assaying methods may be limited.

Accordingly, what are needed are novel microorganisms, such as genetically modified S. lividans, that do not produce undecylprodrodigiosin, actinorhodin, or both antibiotics.

Pseudomonas spp. are also well-characterized laboratory organisms. Pseudomonads are Gram-negative bacteria. They can colonize many niches including soil, fresh water, and biotic and abiotic surfaces. They have large genomes (over 6 Mb) and are know to have rich metabolic diversity, including degradation of xenobiotics, and production of secondary metabolites such as polyketides and non-ribosomal peptides. Importantly, a wide variety of tools have been developed for these organisms (including transformation, conjugation, transposon mutagenesis, and a wide variety of vectors and reporter systems), making their genetic manipulation relatively straightforward. Thus, the development of a pseudomonad host would be a valuable addition to our host repertoire since it would allows to express small molecules such as polyketides and non-ribosomal peptides in a bacterium amenable to FACS sorting, and therefore, high throuput genetic prescreens.

To create such microorganisms, what are also needed are novel and useful vectors that permit the removal or replacement of chromosomal DNA segments in the microorganisms, and yet do not insert a selectable marker gene, such as an antibiotic resistance gene, into the microorganism as a side product.

In addition, what are also needed are novel vectors that can be used to introduce large pieces of DNA (up to 300 kb) into bacteria, such that these pieces of DNA become integrated into the bacterial chromosome to enhance their stability, and novel methods that allow high-throughput transfer of these large DNA segments into bacteria.

The citation of any reference herein should not be construed as an admission that such reference is available as “Prior Art” to the instant application.

### SUMMARY OF THE INVENTION

In a first aspect, the present invention extends to a vector for introducing genetically unmarked mutations in the chromosome of a unicellular, particularly bacterial, host. An example of such bacterial hosts include, but certainly are not limited to, Streptomyces and Pseudomonas spp. Such a vector of the present invention comprises an origin of replication, a counterselectable marker for bacteria, and a selectable marker for bacteria, wherein the selectable marker is excised after crossover in the unicellular host. The use of a vector of the present invention results in no vector sequence or drug resistance marker remaining in the genome of the unicellular host after a second crossover event.

Numerous origins of replication are well known to those of ordinary skill in the art, and have applications herein. A particular origin of replication having applications herein comprises the temperature-sensitive origin of replication of the S. ghanaensis plasmid pSG5. Likewise, a wide variety of genes encoding a counterselectable marker can be used in a vector of the present invention. A particular example of such a counterselectable marker comprises rpsl. A particular example of a selectable marker usable in the present invention is a gene that confers thiostepton resistance to the host.

Furthermore, particular examples of vectors of the present invention comprise pGM160rpsL14, which was deposited pursuant to the requirements set forth in the Budapest Treaty at the American Type Culture Collection, 10801 University Boulevard, Manassas, Va. 20110-2209, United States of America (hereinafter referred to as “ATCC”) on Nov. 9, 2001, and has been assigned ATCC accession number PTA-3850. Another example of a particular vector of the present invention is pSrpsL6, which was deposited pursuant to the Budapest Treaty at ATCC on Dec. 13, 2001, and has been assigned ATCC accession number PTA-3849. These examples are described in detail infra.

Furthermore, it is an object of the present invention to provide novel genetically modified Streptomyces strains that do not produce undecylprodigiosin, actinorhodin, or both. These compounds are pigmented antibiotics that can interfere with a variety of assay methods. Accordingly, the present invention extends to a genetically modified S. lividans strain that is unable to produce actinorhodin. The genetic modification in such a strain of the present invention comprises the unmarked deletion of the act gene cluster in the genome of this strain. A particular example of such a strain of the present invention is described infra. This strain was deposited on Nov. 9, 2001 with ATCC pursuant the Budapest Treaty, and has been assigned ATCC accession number PTA-3847.

In another embodiment, the present invention extends to a genetically modified S. lividans strain that is unable to produce undecylprodigiosin, wherein the modification comprises the deletion of the red gene cluster in the genome of the strain. Such a strain of the present invention was deposited with ATCC on Nov. 9, 2001, and has been assigned ATCC accession number PTA-3848.

Furthermore, the present invention extends to a genetically modified S. lividans strain that is unable to produce actinorhodin and undecylprodrodigiosin, wherein the modification comprises the deletion of the act and red gene clusters in the genome of the strain. These metabolites are pigmented, and can interfere with a variety of assay methods. However, in a strain of the present invention, such metabolites are not produced. Thus, strains of the present invention lend themselves to use in assays that could not be used with heretofore known strains.

In another embodiment, the present invention extends to various plasmids and methods for transferring a large DNA sequence into a bacterial cell, particularly a Stroptomyces or Pseudomonas spp. As explained above, heretofore known methods for transferring large DNA sequences into bacterial cells such as Streptomyces are limited because generally, they involve difficult and laborious procedures, such as polyethylene glycol-mediated protoplast transformation, that are not amenable to automation. A much-preferred method of transferring DNA into Streptomyces, conjugation between Escherichia coli and Streptomyces, as currently practiced in the field, is unable to transfer DNA sequences greater than 45 kilobases. However, the vectors and methods of the present invention readily permit the transfer of large DNA sequences. In one example, a DNA sequences of 100 kilobases, is transferred from E. coli to S. lividans. Furthermore, once transferred into a Streptomyces cell, the invention permits the integration of this DNA into the Streptomyces chromosome, to enhance the stability of the DNA in its new host. Thus, the present invention lends itself extremely well to various assays, particularly high-throughput screening of proteins and metabolites encoded by the large DNA sequence.

One element of the present invention that is important to such a transfer is a cassette that comprises:

(a) a first loxP site;

(b) a DNA sequence that encodes for an integrase operably associated with a promoter;

(c) an attP site for integration of a DNA sequence contained in the vector into the bacterial chromosome;

(d) an origin of transfer for conjugation; and

(e) a second loxP site, such elements (b), (c) and (d) lay between elements (a) and (e).

In a particular embodiment, the integrase gene and the attP site for integration are from phage ΦC31.

Furthermore, the present invention extends to a cassette as described above, which further comprises an antbiotic resistance gene. Numerous antibiotic resistance gene readily well-known to those of ordinary skill in the art have applications herein.

In another embodiment, the cassette is inserted into a vector comprising a counterselectable marker, the Bacillus subtilis sacB gene. This vector can be used as a donor in an in vitro cre-loxP recombination reaction to transfer the cassette to a variety of additional vectors.

Furthermore, the present invention extends to two bacterial artificial chromosome (BAC) vectors that can be used to clone large DNA fragments and transfer those fragments into a bacterial cell. Such BACs of the present invention comprise a BAC vector that can replicate in E. coli, and a cassette of the present invention to allow conjugal transfer and integration into the bacterial chromosome. A particular example of a bacterial artificial chromosome of the present invention includes pMBD13, which was deposited on Dec. 13, 2001 with ATCC pursuant to the Budapest treaty and has been assigned ATCC accession number PTA-3854. Another example of a bacterial artificial chromosome of the present invention is pMBD14, which deposited on Nov. 9, 2001 with ATCC pursuant to the Budapest Treaty, and has been assigned accession number PTA-3855.

It is also an object of the present invention to provide genetically modified bacterial species, particularly Streptomyces and Pseudomonas spp., which can accept the BACs of the present invention and allow conjugal transfer of the BAC and integration into their bacterial chromosomes.

Naturally, the present invention further extends to a method for inserting a large DNA sequence into bacteria that utilizes a BAC of the present invention. As explained above, the successful insertion of such a DNA sequence into bacterial species readily has application in a wide variety of assays, including high throughput screening. Hence, the present invention extends to a method for transferring a large DNA molecule from E. coli to Streptomyces and/or Pseudomonas. The first step of such a method comprises providing an E. coli cell bearing a bacterial artificial chromosome (BAC) vector, wherein the BAC comprises a BAC vector with a large DNA molecule insertion and a cassette of the present invention. The next step of a method of the present invention is to provide a Streptomyces or Pseudomonas cell. Any strain of Streptomyces or Pseudomonas, in either its wild type form or a genetically modified form, has applications in a method of the present invention. The E. coli cell and the Streptomyces or Pseudomonas cell are then placed in contact so that a conjugative transfer can occur between the two cells in which the BAC is transferred from the E. coli cell to the Streptomyces or Pseudomonas cell. After the conjugative transfer, the Streptomyces or Pseudomonas cell containing the BAC is selected using the antibiotic resistance marker present in the BAC of the present invention. As explained above, the large DNA sequence that can be transferred into a bacterial cell with the present invention can exceed 45 kilobases, and could be as large as 300 kilobases

These and other aspects of the present invention will be better appreciated by reference to the following drawings and Detailed Description.

### BRIEF DESCRIPTION OF THE DRAWINGS

FIG. 1: A schematic view of the gene replacement vectors pSrpsL14 and pSrpsL6, and plasmids pΔact and pΔred used to delete the act and red clusters in S. lividans.

FIG. 2: A schematic view of the method for deletion of an act cluster in the genome of S. lividans.

FIG. 3: A schematic view of the deletion of the act and red gene clusters from the genome of S. lividans.

FIG. 4: A view of the antibiotic production phenotype of S. lividans strains of the present invention.

FIG. 5: A comparison of the views and the HPLC profiles of TK24 and S. lividans ΔactΔred.

FIG. 6a: A schematic view of plasmid pMBD7, pMBD9, and pMBD12 of the present invention.

FIG. 6b: Schematic views of cassettes of the present invention.

FIG. 7: View and HPLC profile of S. lividans conjugated with plasmid pMBD10 of the present invention (negative control) compared with view and HPLC profile of S. lividans conjugated with plasmid pSGran.

FIG. 8: Schematic views of the BAC vectors of the present invention, pMDB13 and pMBD14.

FIG. 9: Schematic view of plasmid pBTP3.

FIG. 10: Schematic views of the process of integrating the ΦC31 (Streptomyces phage) attB site in the chromosome of P. putida.

FIG. 11: Southern analysis showing integration of BamBAC8 into MBD1 chromosome.

FIG. 12: RP-HPLC elution profile showing expression of heterologous molecules in P. putida MBD1.

FIG. 13: Growth plate showing results of high-throughput conjugation.

### DETAILED DESCRIPTION OF THE INVENTION

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994).

Therefore, if appearing herein, the following terms shall have the definitions set out below.

A “vector” is a replicon, such as plasmid, phage, bacterial artificial chromosome (BAC) or cosmid, to which another DNA segment may be attached so as to bring about the replication of the attached segment. A “replicon” is any genetic element (e.g., plasmid, chromosome, virus) that functions as an autonomous unit of DNA replication within a cell, i.e., capable of replication under its own control.

As used herein, a “cassette” refers to a segment of DNA that can be inserted into a vector by site-specific recombination.

“Site-specific recombination” refers to a recombination process between two DNA molecules that occurs at unique sites of each molecule which are generally 20-30 bases long, called attachment (att) sites. A specialyzed enzyme, the “integrase”, recognizes the two att sites, joins the two DNA molecules and catalyzes a DNA double-strand breakage and rejoining event that results in the integration of one of the DNA molecules into the other

As used herein, the term “Recombinant Bacterial Artificial Chromosome” (BAC) refers to a BAC vector containing a large insertion of DNA, up to 300,000 bases in size. Once the Recombinant BAC DNA has been introduced into a host bacterium, copies of it can be made.

As used herein, the term “conjugative transfer” refers to the temporary union of two bacterial cells during which one cell transfers part or all of its genome to the other.

As used herein, the term “large DNA fragment” refers to a piece of DNA that has an approximate size ranging from about 45 kilobases to about 200 kilobases.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Heterologous” DNA refers to DNA not naturally located in the cell, or in a chromosomal site of the cell. Preferably, the heterologous DNA includes a gene foreign to the cell.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

“Homologous recombination” refers to an enzymatic process by which DNA, that could comprise a vector, may be inserted into a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

As used therein, a “genetically unmarked mutation” refers to a mutation that does not include a selectable marker, such as an antibiotic resistance gene.

A “counterseletable marker” refers to a gene that, under appropriate growth conditions, promotes the death of the microorganism harboring it.

A DNA “coding sequence” is a double-stranded DNA sequence that is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus.

Transcriptional and translational control sequences are DNA regulatory sequences, such as promoters, enhancers, terminators, and the like, that provide for the expression of a coding sequence in a host cell.

A “promoter sequence” or “promoter” can be used interchangeably, and refer to a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which is then trans-RNA spliced and translated into the protein encoded by the coding sequence.

The present invention may be better understood by reference to the following non-limiting Example, which is provided as exemplary of the invention. The following Example is presented in order to more fully illustrate the preferred embodiments of the invention. It should in no way be construed, however, as limiting the broad scope of the invention.

### EXAMPLES

### Materials and Methods

Bacterial Strains and Plasmids.

The strains and plasmids used in this study are listed in Table 1.

TABLE 1
Plasmids and strains.

| Plasmid | Description | Source |
|---|---|---|
| pBKII SK⁻ | General cloning vector; bla (Amp^{R}) | Stratagene |
| pCR4Blunt-TOPO | PCR product cloning vector | Invitrogen |
| pDNR-1 | Cre-lox donor vector; bla (Amp^{R}), sacB (sucrose^{S}) | Clonetech |
| PBKrpsL | 1.3 kb fragment containing S. coelicolor rpsL | this work |
| | cloned into EcoRI of pBKII SK⁻ | |
| pGM160 | Ts Streptomyces pSG5 ori; aac1, bla (Amp^{R}), tsr | W. Wohlleben |
| | (Thio^{R}) | (15) |
| pSrpsL6/14 | 1.3 kb EcoRI fragment of pBKrpsL cloned into | this work |
| | pGM160; bla (Amp^{R}) tsr (Thio^{R}); rpsL (Strep^{R}) | |
| PDact | PSrpsL14 derivative containing actVB and actVI | this work |
| | fragments of S. lividans act cluster; bla (Amp^{R}), tsr | |
| | (Thio^{R}); rpsL⁺ (Strep^{S}) | |
| pTOPO-TK3 | redD PCR product cloned in pCR4Blunt-TOPO | this work |
| pBK-TK4 | SC10A5.02 PCR product cloned in pBKII SK⁻ | this work |
| PDred | PSrpsL6 derivative containing the redD fragment | this work |
| | from pTOPO-TK3, and the SC10A5.02 fragment | |
| | of pBK-TK4 | |
| pOJ446-22-16 | Cosmid encoding the granaticin gene cluster from | D. Hoffmeister |
| | S. violaceoruber Tu22 | (1) |
| pOJ436 | Streptomyces conjugative cloning vector; aac(3)IV | P. Jeannin |
| | (Apra^{R}) | (Aventis)(2) |
| pBeloBac11 | BAC cloning vector; Chlor^{R}; single copy | (22) |
| pMBD7 | 6.6 kb SpeI-DraI fragment of pOJ436 cloned in | this work |
| | pDNR1; Apra^{R}; Amp^{R} | |
| pMBD9 | PMBD7 derivative with BstXI site removed; | this work |
| | Apra^{R}; Amp^{R} | |
| pMBD12 | PMBD9 derivative with BamHI site removed; | this work |
| | Apra^{R}; Amp^{R} | |
| pMBD10 | PMBD7 Streptomyces cassette in pBeloBac11; | this work |
| | Apra^{R}; Chlor^{R}; single copy | |
| pMBD13 | PMBD9 Streptomyces cassette in pBTP3; Apra^{R}; | this work |
| | Chlor^{R}; high copy | |
| pMBD14 | PMBD12 Streptomyces cassette in pBeloBac11; | this work |
| | Apra^{R}; Chlor^{R}; single copy | |
| pBTP3 | modified BAC vector; Chlor^{R}; high copy | I. MacNeil |
| | | (Aventis) |
| MG1.1 | Antibacterial soil BAC clone | I. MacNeil |
| | | (Aventis) (11) |
| pSMG1.1 | PMBD9 Streptomyces cassette in MG1.1; Chlor^{R}; | this work |
| | Apra^{R} | |
| PGran | 38 kb granaticin cluster from pOJ446-22-16 cloned | this work |
| | into pBTP3 | |
| PSGran | PMBD9 Streptomyces cassette in pGran | this work |
| p1000 | Source of ΦCTX (P. aeruginosa phage) attP site | Justus-Liebig- |
| | | Universitaet |
| pIHB | pUC plasmid encoding the ΦCTX integrase | Justus-Liebig- |
| | | Universitaet |
| pCR-BluntII Topo | PCR cloning vector | Invitrogen |
| pMON1522 | Source of DAPG cluster | U.S. Dept of |
| | | Agriculture |
| pSDAPG | 6.5 kb XbaI-EcoRI fragment of pMON1522, | this work |
| | containing DAPG cluster cloned into BstX1 cut | |
| | pMBD13 | |
| pTOPOattB | 939 bp PCT fragment of ΦC31 attB sequence of S. | this work |
| | lividans cloned into pCR-BluntII Topo | |
| p2.10 | 1.1 kb PstI fragment from p1000 containing ΦCTX | this work |
| | attP site cloned into PstI site of pTOPOattB | |
| E. coli DH10B | General cloning strain | Gibco/BRL |
| E. coli | High efficiency donor strain for conjugative | P. August |
| ET12567/pUB307 | transfer into Streptomyces; Chlor^{R}; Kan^{R}; dam⁻; | (7) |
| | dcm⁻ | |
| E. coli | Donor strain for conjugative transfer into | this work |
| DH10B/pUB307 | Streptomyces; Chlor^{R}; Kan^{R}; | |
| S. coelicolor A2(3) | RpsL⁺ | John Innes Centre |
| S. lividans TK24 | RpsL (Strep^{R}); act⁺; red⁺ | John Innes Centre |
| S. lividans Δact | TK24 derivative with a deletion of the act cluster; | this work |
| | red⁺ | |
| S. lividans Δred | TK24 derivative with a deletion of the red cluster; | this work |
| | act⁺ | |
| S. lividans ΔactΔred | TK24 derivative with deletions of the act and red | this work |
| | clusters | |
| P. putida KT-2440 | laboratory strain of P. putida | Dartmouth |
| | | University |
| P. fluorescens | source of 2,4-diacetylphloroglucinol cluster | ATCC |
| ATCC49323 | | |
| P. putida MBD1 | Kan^{R}, ΦC31 attB site⁺ | this work |

pMBD12 was deposited on Nov. 9, 2001 with ATCC pursuant to the Budapest Treaty, and has been assigned ATCC accession number PTA-3853.

Media and Growth Conditions.

E. coli and P. putida were grown in LB. S. lividans was grown in R2, R2YE, R5, GYM, or YEME, as indicated (9). Antibiotic concentrations are given in micrograms/ml.

Plasmid Construction.

Standard methods were used for DNA isolation and plasmid construction (9, 19). PCR was performed using Vent polymerase (New England Biolabs) according to manufacturer's instructions, with addition of 7.5-10% DMSO.

a. pSrpsL.

The gene replacement vector pSrpsL was constructed as follows. The wild type rpsL gene was PCR-amplified from S. coelicolor A2(3) using primers rpsL5′ (5′GGAATTCCTTCGTCCGCCACGACACG3′(SEQ ID NO: 1)) and rpsL3′ (5′GGAATTCCGTCTTGCCCGCGTCGATG3′(SEQ ID NO: 2)). The 1.3Kb rpsL fragment was digested with EcoRI (restriction sites underlined) and cloned into the EcoRI site of pBKII SK⁻ (Stratagene), yielding pBKrpsL122. The rpsL fragment of pBKrpsL122 was isolated after EcoRI digestion and cloned into the EcoRI site of pGM160 (15), resulting in pSrpsL14 and pSrpsL6, depending on the orientation of the insert. pSrpsL6 was deposited on Nov. 9, 2001 with ATCC pursuant to the Budapest Treaty, and has been assigned ATCC accession number PTA-3849. PSrpsL14 was deposited on Dec. 13, 2001 with ATCC pursuant to the Budapest Treaty, and has been assigned ATCC accession number PTA-3850.

b. pΔact18.

The actVIBA genes (left end of the act cluster (6)) were PCR-amplified from S. lividans TK24 using the primers

(SEQ ID NO:3))

| | |
|---|---|
| actVI5′ | (5′GAAGATCTTCGGCAGCGCGTCAGGGTGTCA3′ and |

(SEQ ID NO:4))

| | |
|---|---|
| actVI3′ | (5′GGAATTCCTACTGCCTGGTGCTCACCGTCCAC3′, |

and digested with BglII and EcoRI. The actVB ORF11 and ORF12 genes at the right end of the act cluster (13) were also PCR-amplified from TK24 using primers

| | |
|---|---|
| act.lysR25′ | (SEQ ID NO:5)) |

(5′GGAATTCCACGAGGGTGGTTGGCGTCGGAACAAGGC3′ and

| | |
|---|---|
| act.lysR23′ | (SEQ ID NO:6)) |

(5′CGGGATCCCAGGAAGCACAGGACGCCGAGGACGAAC3′

and digested with EcoRI and BamHI. The actVI and actVB fragments were ligated with pSrpsL14 that had been digested with BamHI and BglII and dephosphorylated with SAP. The resulting plasmid, pΔact18, was used to delete the act cluster from S. lividans.

3. pΔred.

The redD gene was PCR-amplified from TK24 using primers redD5′.Xover

| | |
|---|---|
| (5′GACGGCCAAGCTTCCTCGACCTTGTGGACCTCGTCGGTGCGCATCA3′ | (SEQ ID NO:7)) |

and redD3′.Xover

| | |
|---|---|
| (5′GATCATCGGGTCGTCTGTTTAAACGGTCGTCAGGCGCTGAGCAGGCTGGTGT3′. | (SEQ ID NO:8)) |

The PCR product was cloned into pCR4-Blunt-TOPO (Invitrogen), resulting in pTOPO-TK3. The S. lividans homolog of SC10A5.02 (chosen as the right end of the red cluster) was amplified using primers

| | | |
|---|---|---|
| red.oxidase.5′.Xover | (5′CGCCTGACGACCGTTTAAACAGACGACCCGATGATCCCCAACCAGTGG3′ and | (SEQ ID NO:9)) |
| red.oxidase.3′ | (5′CGGGATCCCGCGGGGTCAGTACACGTAGGGGACGAACTTC3′, | (SEQ ID NO:10)) |

and cloned into the EcoRV site of pBKII SK⁻ (Stratagene) to yield pBK-TK3. pΔred was created by ligating the redD fragment (as a 1.9 kb HindIII-PmeI fragment of pTOPO-TK3) and the SC10A5.02 homolog fragment (a 1.9Kb PmeI-BamHI fragment from pBK-TK4) into pSrpsL6 that had been cut with HindIII and BamHI. pΔred was used to delete the red cluster from S. lividans.

4. pMBD7, pMBD9, and pMBD12.

pMBD7 was constructed by cloning a 6.6 kb SpeI-DraI fragment of pOJ436 (2) into a 4.1 kb XbaI-partial PvuII fragment of pDNR-1 (Clonetech). pMBD7 was deposited on Nov. 9, 2001 with ATCC pursuant to the Budapest Treaty, and has been assigned ATCC accession number PTA-3851. Transformants in DH10B were selected on LB Amp₁₀₀ Apra₅₀ plates and tested for sensitivity to 7% sucrose (conferred by the intact sacB gene in the partial PvuII fragment) prior to restriction analysis. pMBD9 is a derivative of pMBD7 in which a BstXI site at the end of the aac(3)IV gene has been removed by digestion with BstXI, blunting of the ends with T4 DNA polymerase, followed by religation. It was deposited on Nov. 9, 2001 with ATCC pursuant to the Budapest Treaty, and has been assigned ATCC accession number PTA-3852. pMBD12 is a derivative of pMBD9 in which the unique BamHI site has been removed by the method described above for pMBD9.5. pMBD12 has also been deposited with ATCC pursuant to the Budapest Treaty on Nov. 9, 2001, and has been assigned ATCC accession number PTA-3853.

5. pGran.

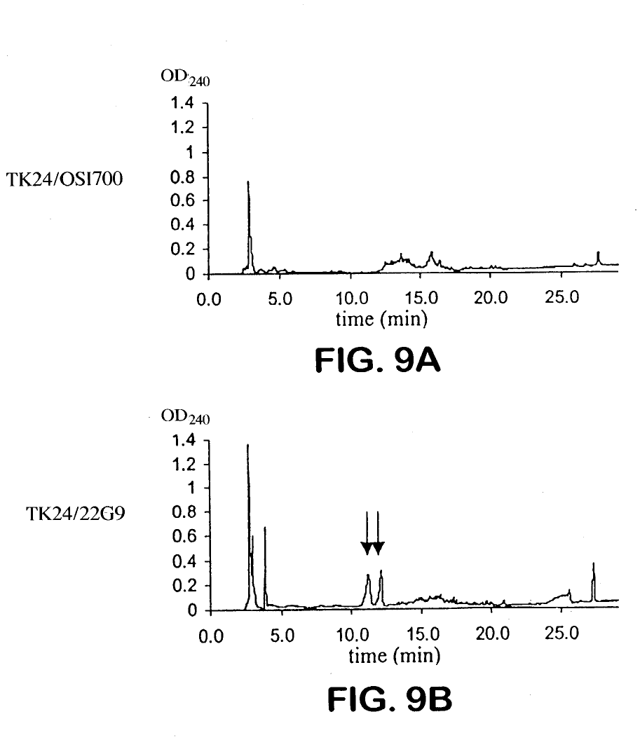

The 38.2 kb EcoRV fragment of pOJ446-22-24 containing the granaticin gene cluster from S. violaceoruber Tu22 (1) was cloned into pBTP3, a modified pBeloBac11 containing a pUC vector inserted into the polylinker region and BstXI cloning sites (FIG. 9) by the adapter cloning method. Briefly, pOJ446-22-24 was digested with EcoRV, ethanol precipitated, and ligated to BstX1 adapters (Invitrogen N418-18) in 1× blunt-end ligation buffer (50 mM TrisHCl pH7.8, 50 mM ATP, 10 mM BME, 5 mM MgCl₂), 15% w/v PEG 8000, 400U ligase, incubated at 16° C. overnight. The granaticin-encoding fragment was purified by pulse field electrophoresis in a 1% LMP (0.5×TBE, 0.1s to 35s switch time, 6V, 14° C. for 12 hrs). The gel slice was dialyzed against TE buffer for 2 hrs prior to digestion with Gelase (Epicenter) according to the manufacturer's recommendations, and ligated to 20 ng of BstX1 cut pBTP3 vector (10:1 vector:insert molar ratio) at 16° C. for 6 hrs. After electroporation into ElectroMax DH10B cells, transformants were selected on LB Chlor₁₂ plates.

6. pSDAPG.

A 6.5 kb XbaI-EcoRI fragment containing the locus required for 2,4-diacetylphloroglucinol synthesis (DAPG cluster) was excised from pMON1522, blunted using T4 DNA polymerase, and ligated to BstX1 adaptors (Invitrogen N418-18) in 1× blunt-end ligation buffer (50 mM TrisHCl pH7.8, 50□M ATP, 10 mM BME, 5 mM MgCl₂), 15% w/v PEG 8000, 400U ligase at 16° C. overnight. The DAPG fragment was gel-purified, and ligated to 20 ng of BstX1 cut pMBD 13 vector (10:1 vector: insert molar ratio) at 16° C. for 6 hrs. After electroporation into ElectroMax DH10B, transformants were selected on LB Chlor12.5 plates.

Gene Replacement in S. lividans.

Plasmids pΔact and pΔred were used separately to transform TK24 (Sm^{r}) protoplasts by standard methods (9). Transformants were selected in R2YE containing thiostrepton (Thio₅₀) at 29° C. Individual transformants were submitted to two rounds of growth in YEME Thio₈ for 3-5 days at 29° C., homogenized and plated in R2YE Thio₅₀ at 39° C. to select for single crossover events. Several (4-6) clones selected at 39° C. were grown for 5 days at 39° C. in YEME Thio₈. 100 ml of the culture were used to inoculate 100 ml YEME without Thio. Second crossover events resulting in excision of plasmid sequences were selected by plating on GYM (21) Strep₅₀ at 39° C. Each clone was then tested for Thio sensitivity and pigmented antibiotic production in R2 plates. The presence or absence of each antibiotic cluster on the chromosome was verified by PCR analysis using the following primers.

(SEQ ID NO:11)

| | |
|---|---|
| Δact.1 | (GTGGGTACCCGTGGGTACCTGTGCTGCTTT; |

(SEQ ID NO:12))

| | |
|---|---|
| Δact.2 | (TTGTTGACCAGTACGTCCACCCTGCCGTGC; |

(SEQ ID NO:13))

| | |
|---|---|
| Δact.3 | (AGATGCAGAAGCTGGACGGCCGTGACTTCG; |

(SEQ ID NO:14))

| | |
|---|---|
| Δred.1 | (GGCCCTGGAGGATCTCATCAGCGCGATGTT; |

(SEQ ID NO:15))

| | |
|---|---|
| Δred.2 | (TAGAGGGCGGACATCCCGACGATGGCGAT; and |

(SEQ ID NO:16))

| | |
|---|---|
| Δred.3 | (AGCCGTGGTACGGGCATTCGATGGTGTTGC. |

Engeneering of P. putida MBD1.

The ΦC31 attB sequence of S. lividans was PCR-amplified using primers

| | | |
|---|---|---|
| attB5′ | (ACCATCGTGATCGGCGTGTGC and | (SEQ ID NO:17)) |
| attB3′ | (GCCCGTGATCCCGATGTTCACCGG | (SEQ ID NO:18)) |

and Vent polymerase (NEB). The resulting 939 bp fragment was cloned into pCR-BluntII Topo (Invitrogen) yielding pTOPOattB. Next, a 1.1 Kb PstI fragment from plasmid p1000 containing the ΦCTX (P. aeruginosa phage) attP site (27) was cloned into the PstI site of pTOPOattB. The resulting plasmid, p2.10, was cotransformed with pIHB (a pUC plasmid encoding the ΦCTX integrase (27) into electrocompetent P. putida KT-2440. After electroporation, cells were allowed to recover in 2 ml of SOC at 30° C. prior to selection. Transformants in which p2.10 had integrated at the ΦCTX attB site, were selected on LB kanamycin (25 μg/ml). The presence of the ΦC31 attB site in the P. putida chromosome was verified by Southern hybridization using the 938 bp ΦC31 attB fragment as a probe. The resulting strain was named P. putida MBD1. P. putida MBD1 has also been deposited with ATCC pursuant to the Budapest Treaty on Nov. 7, 2002, and has been assigned ATCC accession number PTA-4787.

Transfer of the CIS Cassettes to BAC Vectors.

The CIS cassettes of pMBD7, pMBD9 and pMBD12 were transferred to BAC vectors by in vitro cre-lox recombination using cre recombinase (Clonetech) according to the manufacturer's instructions. Recombination products were selected after transformation into ElectroMax DH10B (Gibco/BRL) by plating in LB Chlor₁₂, Apra₃₀ agar containing 7% sucrose.

BamBAC Library Construction.

Megabase environmental DNA was isolated from a local soil as previously described (11) The soil megabase DNA plug was dialyzed against 15 ml 1×BamHI buffer with BSA at 4° C. for 1 hr. The gel slice was melted at 65° C. for 5 min, equilibrated to 37° C. for 5 min, and digested with 0.8 units of BamHI at 37° C. for 1 hr. The digestion was stopped by addition of EDTA (50 mM final concentration). After electrophoresis in 1% LMP pfge (0.5×TBE, 0.5s to 35s switch time, 6V, 14° C. for 10 hrs), a gel slice containing 50-100 kb DNA was excised from the gel and digested with gelase (Epicenter). Soil DNA was ligated at 16° C. overnight (10:1 vector:insert molar ratio) with 20 ng of pMBD14 that had been digested with BamHI and dephosphorylated with CIP. Two μl of the ligation were used to electroporate ElectroMax DH10B (Gibco/BRL,0.2 cm cuvette, 2.5KV). Transformants were selected on LB Chlor₁₂ Apra₃₀ plates.

Conjugations for Streptomyces.

Conjugations of individual plasmids were performed essentially as described (9) using ET12567/pUB307 or DH10B/pUB307 as the donor strain. When DH10B/pUB307 was used as a donor, log phase cultures (OD₆₀₀ of 0.5-0.7) were diluted 1:100 for conjugation.

Conjugations for Pseudomonas.

The donor E. coli DH10B pUB307 strain containing the BAC construct to be transferred was grown overnight at 37° C. in LB containing Chlor12.5, Apra30, Kan50. The recipient, P. putida MBD1 was also grown overnight at 30° C. in LB Kan50. Donor and recipient were subcultured 1:100 in fresh medium and grown for 4 hr. The recipient was incubated at 42° C. for 15 min to inactivate restriction enzymes. Donor and recipient are mixed in a 1:3 ratio in an eppendorf tube, centrifuged for 1 min, and resuspended in 50 □l of LB. The mix is placed in a LB agar plate and incubated at 30° C. After 24 hr, cells were scraped from the plate and rsuspended in 1 ml of LB. Dilutions were plated in LB Nal20, Apra25. Exconjugants were picked after 2 day incubation at 30° C.

High Throughput Transfer of Libraries into S. lividans.

High throughput transfer of environmental libraries was performed as follows. Pools of BAC library transformants in DH10B were grown on LB Apra30 Chlor12, and BAC DNA isolated using Quiagen Maxiprep kit. Pooled BAC DNA was used to transform electrocompetent ET12567/pUB307 or DH10B/pUB307. Transformants were picked with a Q-bot robot (Genetix) into 96-well deep plates containing LB Apra30 Chlor12 Kan50 and grown overnight at 37° C. Cultures of ET12567/pUB307 were used directly whereas DH10B/pUB307 cultures were diluted 1:1000 in fresh medium prior to conjugation. A 96-pin stamper was used to deposit 5-10 μl aliquots of the E. coli cultures onto plates of a modified R2 medium (as in (9) but without sucrose) that had been previously spread with pregerminated S. lividans spores (approximately 10⁸ spores per 96-well conjugation). Exconjugants were selected after 24 hr at 30° C. by overlaying with SNA containing Nalidixic acid (100 μg/ml final) and Apra (50 μg/ml final concentration). Exconjugants were replicated onto R5 Nal₁₀₀, Apra₅₀.

High Throughput Transfer of Libraries into P. putida.

A DNA library constructed in pMBD14 is transformed into the E. coli donor strain, DH10B pUB307. Individual clones are picked by a Q-bot into deep 96-well plates containing 2 ml of LB with the appropriate drugs. When they have reached stationary phase, donor cultures are diluted 1:10 into fresh LB without drugs. The same donor dilutions are used for conjugations into S. lividans and P. putida. For P. putida, a 96-pin replicator is used to deliver an aliquot of the donor cultures into 96-well plates containing 50 μl of a P. putida MBD1 exponential culture that has been incubated at 42° C. for 15 min to inactivate restriction systems. The same replicator is used to deliver aliquots of the mixes into an LB Q-bot plate. The plate is incubated overnight at 30° C. P. putida exconjugants containing our library clones are selected by replicating the colonies in the LB plate into an M9 benzoate plate [Espinosa-Urgel, 2000 #113] with Apra25 and Nal20. Only exconjugants can grow in this medium. Colonies of exonjugants are visible after 2-3 days of incubation.

Antibacterial Screens.

Clones were grown on R5 or SSM (9) as indicated. Plates were then overlaid with top agar containing exponentially growing Bacillus subtilis strain BR151/pPL608 (Bacillus Genetic Stock Center, Columbus, Ohio) and incubated overnight at 30° C. followed by several days at room temperature. Clones producing antibacterial activities were identified by a zone of inhibition in the lawn surrounding the clone.

Southern Hybridizations.

Chromosomal DNA was prepared using Dneasy columns (Quiagen). 5 mg of chromosomal DNA digested with HindIII or EcoRi were run per gel. Southern hybridization was performed following standars procedures (19). BamBAC8 plasmid DNA and the gel-purified ΦC3attB fragment from pTOPOattB were used for probes. Probes were labeled with P³²-dCTP using the Readyprime II kit (Amersham). ps Preparation of Extracts and HPLC Analysis for S. lividans.

For the preparation of extracts, S. lividans strains were grown in 25 ml YEME Apra₅₀ at 30° C. for 4 days, or in R5 agar plates, as indicated. Cultures were lyophilized and extracted with MeOH:EtOAc 3:1. The extracts were filtered, concentrated (N2 stream, Pierce Reacti-Therm), cleaned by SPE (Waters SepPak C-18 cartridges: Vac 3 cc, 200 mg), dried under N2 stream, redissolved in MeOH and filtered over a 0.2 μm filter. A Metachem Polaris C-18A column (5μ, 120 A, 4.6×150 mm) was used for analytical HPLC. 25 μl samples were applied to the column. The chromatogram was developed as follows. Solvents: A: H2O/0.08% TFA, B: ACN/0.08% TFA. Gradient: 0 min A:B=95:5, 2 min A:B=95:5, 21 min A:B=2:98, 25 min A:B=2:98, 28 min A:B=95:5, 33 min A:B=95:5. The flow rate was 1.5 ml/min and the absorbance of the effluent at 240 nm and 500 nm, 0.1 AUFS, was recorded.

Preparation of Extracts and HPLC Analysis for P. putida.

Crude extracts were prepared and screened as follows. Exconjugants were picked from the selection plate after conjugation onto a fresh M9 benzoate plate with Nal20 Apra25 using a 96-pin replicator. This second round of growth in the presence of selection is used to eliminate residual donor E. coli. A 96-pin replicator is used again to inoculate shallow 96-well plates containing 150 μl of liquid LB medium supplemented with Fe-Citrate (6 mg/L) without drugs. Other media such as YM, M9 citrate or M9 benzoate can be used. Cultures are grown at 29° C. in a humidified container for 5-7 days. At that time, cultures were dried using a Savant Speed-Vac Plus SC210A. One volume methanol was added to the pellets. After 15 min at room temperature in covered plates, the extracts were removed with a multichannel pipettman avoiding the solid residue. Extracts were split in two and dried to completion in the Speed-Vac prior to assaying. For the antibacterial assay, extracts were resuspended in 145 μl of LB. 5 μl of a 1:10 dilution of an early log phase culture of B. subtilis BR151/pPL608 were added to the resuspended extracts, and the plates were incubated at 37° C. overnight with shaking (250 rpm). Growth of the B. subtilis tester strain was evaluated visually. For antifungal assays, extracts were resuspended in 145 μL of YPD media (10 g Yeast Extract, 20 g Peptone, 20 g Glucose) plus 5 μL of 1:100 diluted Candida albicans ATCC 90028 from a frozen glycerol stock. Plates were incubated at 35° C. overnight. Again, growth of the C. albicans tester strain was evaluated visually.

HPLC analysis of secondary metabolite production in P. putida MBD1:

Liquid and plate cultures of P. putida MBD1 exconjugants containing pMBD14, pSgran, pSMG1.1 or pSDAPG were analyzed for metabolite production. Liquid cultures were grown in 50 ml of YM medium (28) containing 25 μg/ml apramycin for 7 days at 27° C. Solid cultures consisted of 150 mm plates with YM agar containing 25 μg/ml apramycin for 7 days at 29° C. Extracts were prepared and analyzed as follows.

Liquid/Liquid Extracts:

50 ml of each bacterial culture were extracted 2× with 25 ml EtOAc [note: phase separation took very long (>1 h) and was still incomplete; the suspension was broken with Na₂SO₄]. It was dried over Na₂SO₄ and filtered. The solvent was removed (Savant Speedvac SC210A) and it was reconstituted in 1 ml H₂O/CH₃CN (50:50 v/v) containing 0.08% TFA. Samples were filtered (Whatman 4 mm, 0.2 mm PTFE syringe filters) prior to HPLC analysis.

Plate Extracts:

Half of each agar plate was cut into pieces of approximately 0.5 cm³ and transferred to 50 ml tubes. The sample was lyophilized for 48 h (Labconco Freezone 4.5), ground to a fine powder, and extracted 2× with 15 ml EtOAc. The extract was filtered and processed as described above.

HPLC Analysis:

An Inertsil ODS-3 column (5 μm, 150×4.6 mm, GL Sciences) was used for analytical RP-HPLC on a Waters 600 system with 996 PDA detector (210-610 nm, 1.2 nm resolution, Millenium 4.0 software). Mobile phase was 0.08% TFA in water (A) and 0.08% TFA in acetonitrile (B). Elution started with 100% A for 2 min, a linear gradient was run from 0%-100% B in 20 min with a 10 min hold at 100% B. The flow rate was 1 ml/min and the injection volume was 10 μl. Identification of 2,4-diacetylphloroglucinol was based on the UV spectrum (29),

### Results

Construction of S. lividans Strains Deleted for One or Both Gene Clusters Encoding Pigmented Antibiotics.

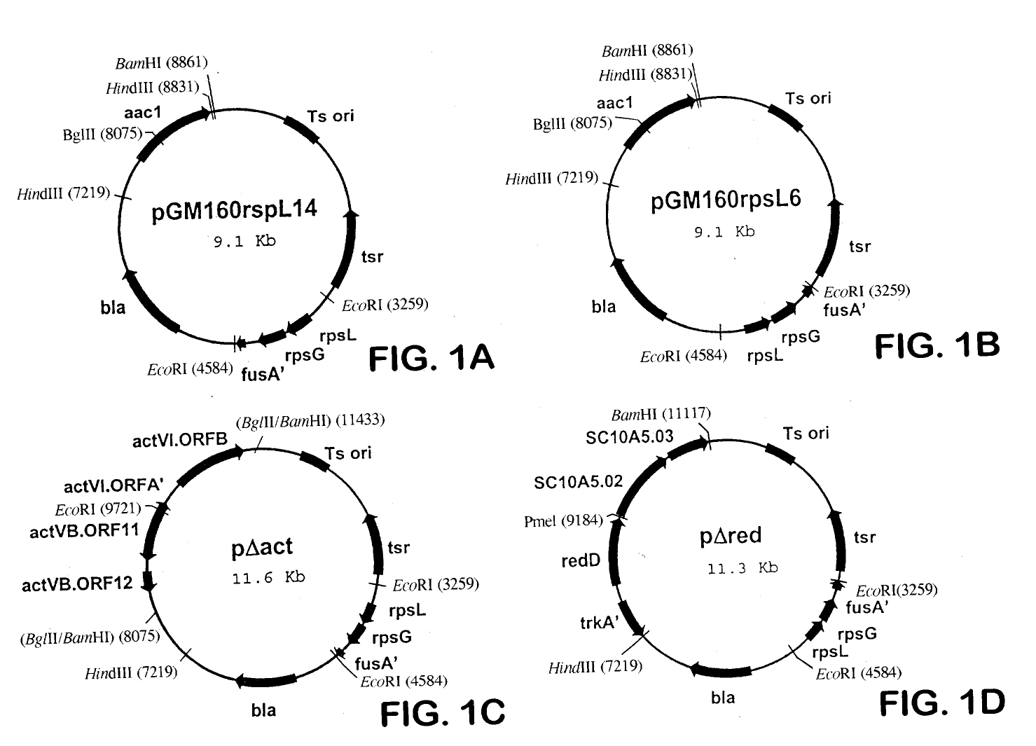

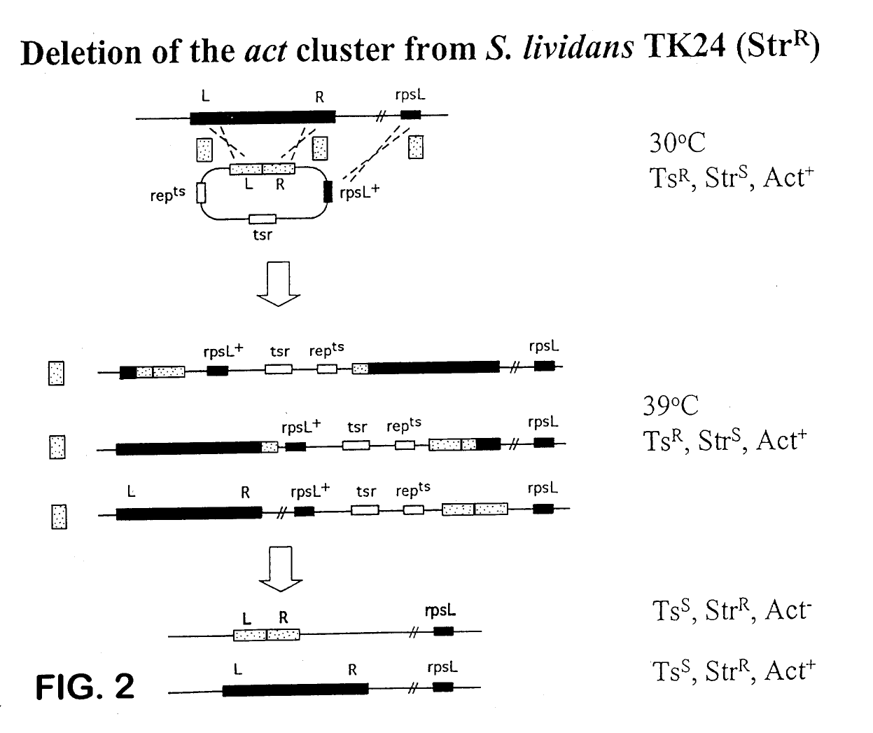

The method used for the construction of the deletion strains was a positive selection of unmarked allelic exchange mutants. This method employs a two-step strategy that combines the use of a temperature-sensitive replicon and a counterselectable marker (reviewed in (17)). It has been shown previously that in S. roseosporus (8), as in other bacteria, the wild type rpsL gene is counterselectable in a Strep^{R} background, since it confers dominant sensitivity to Strep. However, a novel gene replacement vector of the present invention, pSrpsL (FIG. 1), contains the wild type rpsL gene of S. coelicolor A2(3) cloned into pGM160, a shuttle vector with the SG5 ori, which is naturally temperature-sensitive for replication in S. lividans (15). The method for selection of unmarked mutations used herein is analogous to that described for Mycobacterium tuberculosis in U.S. Pat. No. 6,096,549 (16), which is hereby incorporated by reference in its entirety. As an example, the method of deletion of the act cluster is shown in FIG. 2. First, the actVIAB genes (left end of the cluster (6)) and the actVB ORF11-12 genes (right end of the cluster (13)) were cloned in the proper orientation in the gene replacement vector, resulting in pΔact (FIG. 1). pΔact was transformed into TK24, and transformants were selected by resistance to Thio at 29° C., the permissive temperature for plasmid replication. Single crossover events resulting in integration of the plasmid into the chromosome were selected with Thio at 39° C. The three possible integration products are shown in FIG. 2. After a round of growth in liquid medium at 39° C. without antibiotic selection, those cells that have undergone a second crossover event leading to the excision and loss of the plasmid-borne rpsL gene were selected by plating in Sm medium at 39° C. Three out of twelve clones screened by PCR had the unmarked deletion of the act cluster, while the remaining showed the pattern predicted for an intact act cluster.

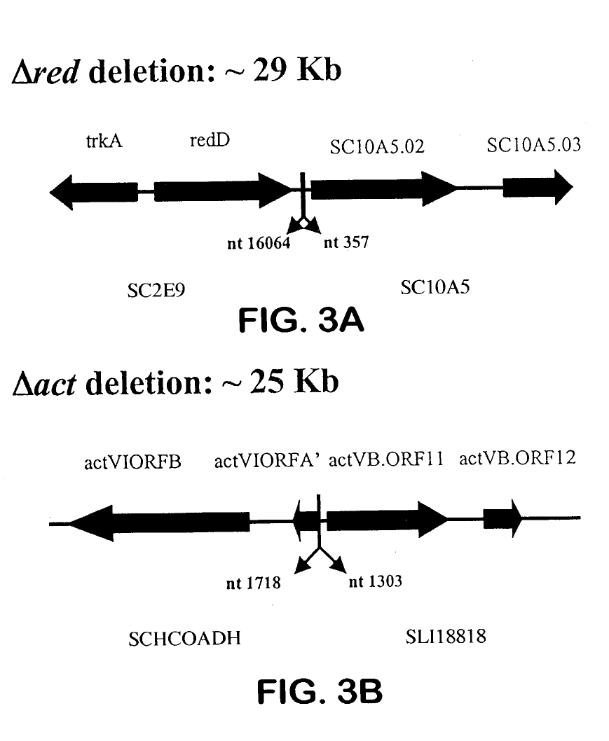

The red cluster is not as well characterized as the act cluster, but it is known that in S. coelicolor the red genes are clustered in a region of approximately 37 kb, with the pathway-specific regulator, redD, at one end (5, 12). The data of the S. coelicolor sequencing project (http://www.sanger.ac.uk/Projects/S coelicolor/) was used to define the limits of the red cluster deletion. redD, as well as redX, redY and redZ, are located on the right end (5 Kb) of the S. coelicolor cosmid 2E9 (Genbank accession number AL021530). The cluster extends through the 24 kb of the overlapping cosmid 3F7 (Genbank accession number AL021409), which encodes putative biosynthetic enzymes such as polyketide and peptide synthetases, into the next cosmid, 10A5 (accession number AL021529). SC10A5.02, which encodes a probable oxidase (the last clearly recognizable putative enzyme in the pathway), was chosen as the right end of the red cluster deletion. The cluster, however, could extend as far as 11 Kb into cosmid 10A5, where a putative antibiotic transport protein (SC10A5.10c) is located. Sequence analyses suggest that the genes in this region are likely to be involved in transport and resistance, and therefore unlikely to interfere with heterologous natural product expression. Thus, S. lividans redD and SC10A5.02 homologs were cloned into the gene replacement vector to create pΔred. This plasmid was used to delete the red cluster in TK24 and S. lividans Δact by the method described above, yielding S. lividans Δred and S. lividans ΔactΔred, respectively. S. lividans Δact, S. lividans Δred, and S. lividans ΔactΔred were deposited with ATCC on Nov. 9, 2001 pursuant to the Budapest Treaty, and have been assigned ATCC accession numbers PTA-3847, PTA-3848, and PTA-3846, respectively. The presence of the act and/or red cluster deletions in the new strains was verified by PCR analysis. The limits of the resulting chromosomal deletions are shown in FIG. 3.

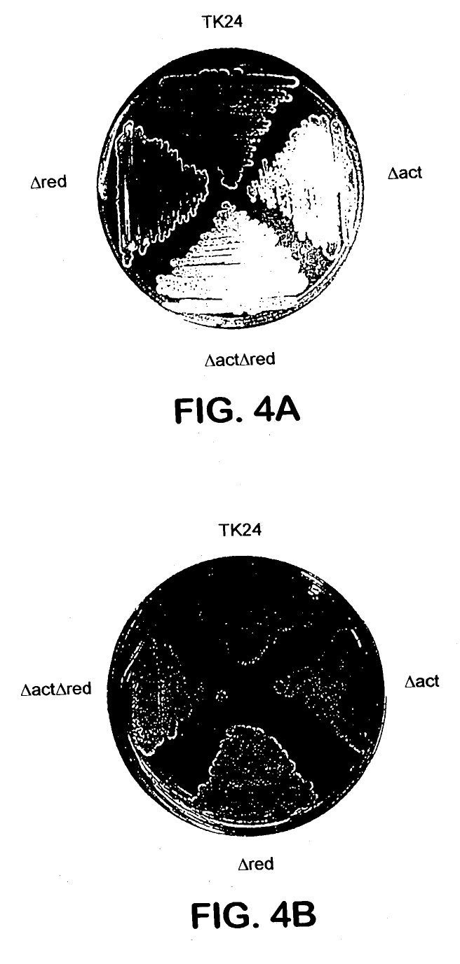

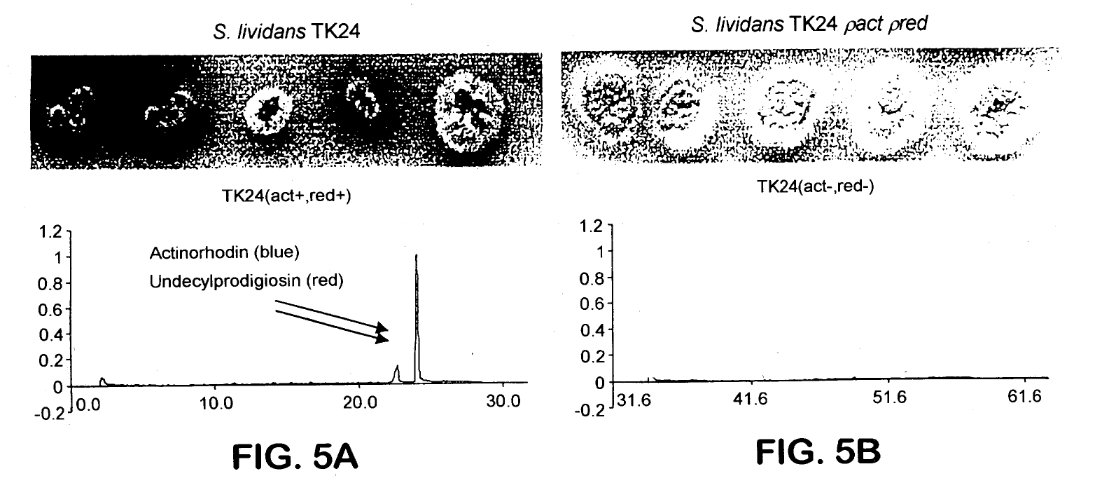

The antibiotic production phenotype of the new strains is shown in FIG. 4. All three new strains grow and sporulate as well as the parental TK24. S. lividans ΔactΔred, which does not produce actinorhodin or undecylprodigiosin has applications as a host for heterologous natural product expression and analysis. A comparison between the HPLC profiles of TK24 and S. lividans ΔactΔred is shown in FIG. 5.

Construction of Plasmids Containing the Cassettes of the Present Invention.

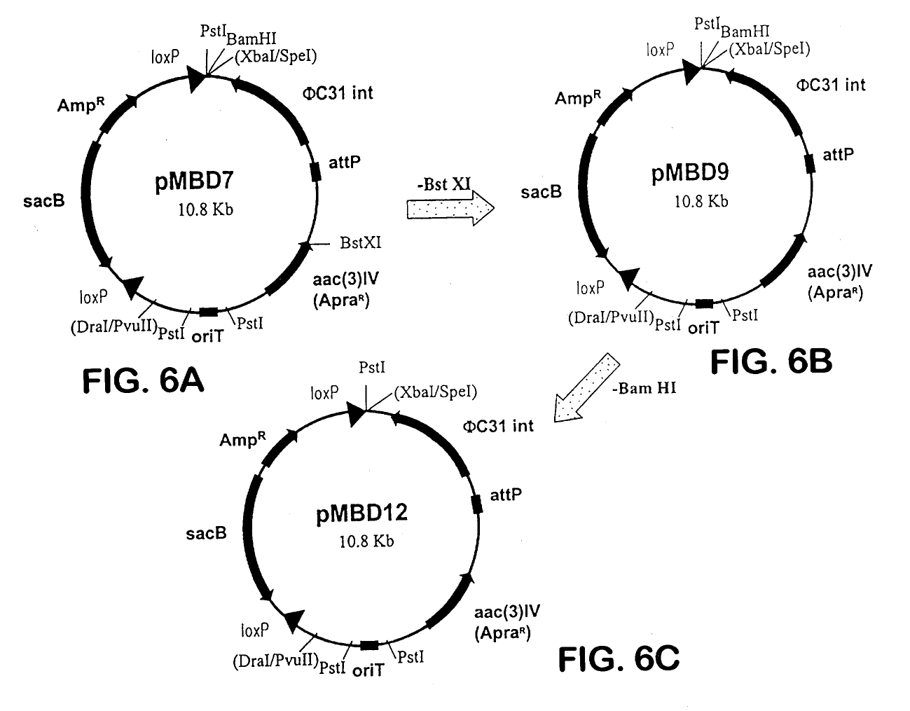

A new series of ΦC31-based vectors has been constructed in which all the elements required for conjugative transfer of DNA into Streptomyces and subsequent integration of the DNA into the chromosome are flanked by two loxP sites, and can thus be efficiently transferred to any loxP-containing plasmid by in vitro cre-lox recombination (20). First, a fragment of pOJ436 (2) that contained the oriT of the self-transmissible plasmid RK4, the ΦC31 integrase and attachment site, and an Apra^{R} marker was cloned between the loxP sites of pDNR-1. This new plasmid, pMBD7 (FIG. 6A), was conjugated into S. lividans with similar efficiency to that of the parent plasmid, pOJ436 (10⁻⁵-10⁻⁶ exconjugants per recipient under conditions set forth herein), using E. coli strain ET12567/pUB307 (7) as the donor. Two derivatives of pMBD7, in which restriction sites were removed, were constructed in order to facilitate further manipulations: pMBD9, deleted for the BstXI site, and pMBD12, deleted for both the BstXI and BamHI sites (FIG. 6A).

The DNA segments flanked by loxP sites in pMBD7, pMBD9 and pMBD12 can be easily transferred to any loxP-containing vector by in vitro cre-lox recombination. These regions have been named, CIS7, CIS9, and CIS12 cassettes, respectively, wherein CIS stands for “Conjugative and Integrative into Streptomyces.” All three cassettes contain the following (see FIG. 6B):

one loxP site at each end of the cassette

the int gene (encoding the integrase) and attP site (for integration in the Streptomyces chromosome) from bacteriophage ΦC31.

the aac(3)IV gene which confers apramycin resistance in E. coli and Streptomyces.

the oriT region of the IncP plasmid RK2 for conjugal transfer into Streptomyces.

Insertion of the CIS Cassettes into BAC Vectors by in Vitro Recombination

The CIS9 and CIS12 cassettes were moved to different BAC plasmids using in vitro cre-lox recombination, and the resulting plasmids were transformed into E. coli strain ET12567/pUB307 for conjugation into S. lividans. The results of these experiments are shown in Table 2.

TABLE 2
Conjugation efficiencies pMBD vectors and large insert shuttle BAC
plasmids.

| | | Conjugation |
|---|---|---|
| Plasmid | Description | efficiency |
| PMBD7 | high copy; contains Streptomyces cassette | 10⁻⁵-10⁻⁶ |
| PMBD9 | high copy; BstXI site removed from pMBD7 | 10⁻⁵-10⁻⁷ |
| PMBD10 | single copy; pMBD9 cassette in pBeloBac11 | 10⁻⁴-10⁻⁵ |
| PSMG1.1 | single copy; pMBD9 cassette in MG1.1 (27 kb | 10⁻⁴-10⁻⁵ |
| | antibacterial insert) | |
| PMBD13 | high copy; pMBD9 cassette in pBTP3 | 10⁻⁶-10⁻⁷ |
| PSGran | single copy; pMBD9 cassette in pGran (38 Kb | 10⁻⁴-10⁻⁵ |
| | insert containing granaticin cluster) | |

pMBD10, a derivative of the single copy E. coli vector pBeloBac11 (22), was conjugated into Streptomyces with high efficiency (10⁻⁴-10⁻⁵, approximately 10 fold higher than the high copy number parent vector pMBD9). Similar high efficiency was measured for the conjugal transfer of the BAC plasmids pSMG1.1 (with a 27 kb soil DNA fragment encoding antibacterial activities in E. coli (11)), and pGran (which encodes a 38 kb fragment containing the granaticin gene cluster of S. violaceoruber Tu22 (1)). These results demonstrate that the CIS cassettes can confer all the functions required for efficient mobilization of single copy BAC vectors with inserts of at least 38 kb.

Heterologous Antibiotic Expression from the New Recombinant BAC Vectors in S. lividans ΔactΔred.

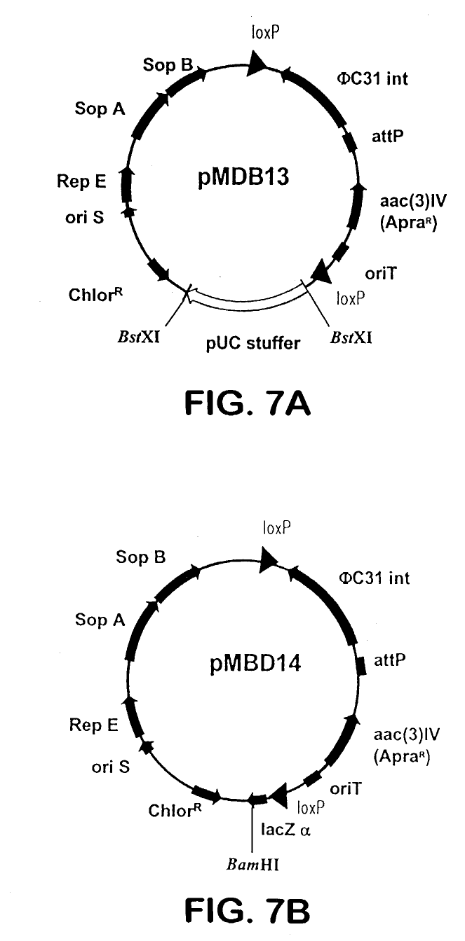

Conjugation of the pGran BAC plasmid containing the granaticin cluster from S. violaceoruber Tu22 (1) into the unpigmented S. lividans ΔactΔred strain led to production of purple pigment that was clearly detectable, both visually and by HPLC, in the otherwise unpigmented background (FIG. 7). The absence of endogenous pigmented antibiotic in S. lividans ΔactΔred also allowed the clear detection of the antibiotic activity of granaticin against Bacillus subtilis strain BR151/pPL608 (data not shown). 100% of the exconjugants tested produced the pigment, indicating that the new vectors are stable in E. coli and S. lividans. Importantly, this result confirms that vectors of the present invention permit heterologous biosynthetic clusters to be efficiently introduced into the unpigmented S. lividans strain by conjugation, and that these clusters can be expressed from the chromosome, leading to clearly detectable antibiotic production by the exconjugants.

An additional gene cluster encoded on plasmid pSMG11 was also tested for antibiotic production in S. lividans strain ΔactΔred. In E. coli, expression of this cluster (MG1.1) leads to the production of blue pigment and antibacterial activity (11). However, the cluster produced neither pigment nor detectable antibacterial activity in strain S. lividans ΔactΔred. Furthermore, no new molecules were detected by HPLC analysis of extracts of S. lividans containing pSMG1.1. These results confirm expected differences in E. coli and S. lividans expression properties, and underscore the importance of screening environmental DNA libraries in multiple hosts.

New Shuttle BAC Vectors for Library Construction.

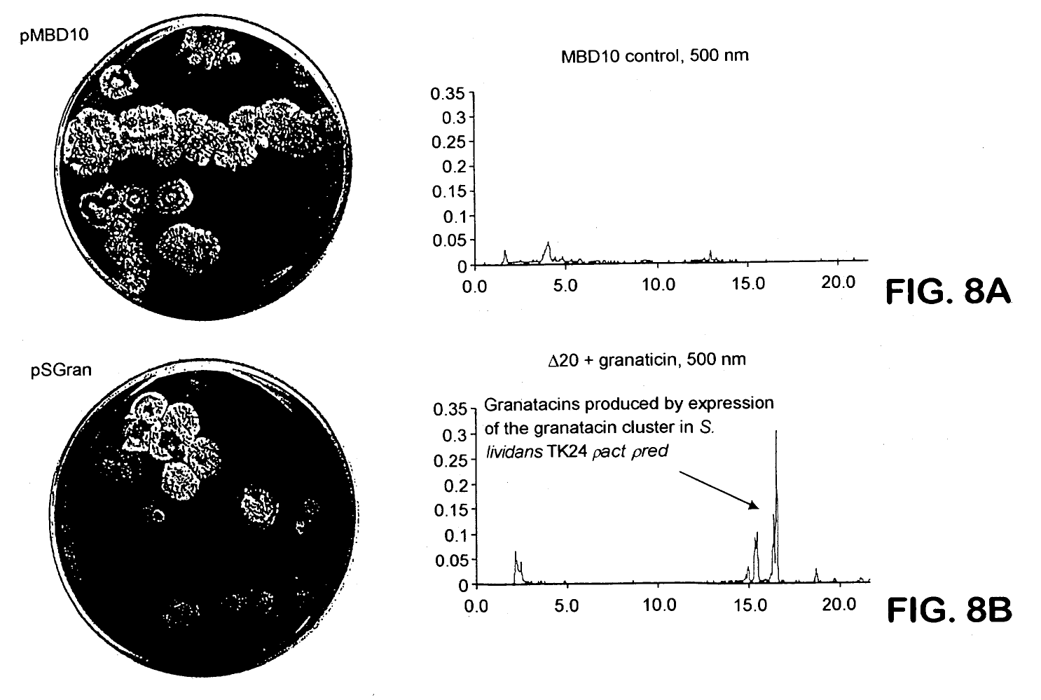

Two new shuttle BAC vectors have been constructed that are useful for the construction of large insert DNA libraries (FIG. 8): pMBD13, in which the CIS9 cassette was inserted into pBTP3 a pBeloBac11 derivative suitable for BstXI adaptor cloning (FIG. 9), and pMBD14, which contains the CIS12 cassette recombined into pBeloBac11. In this latter construct, a unique BamHI site within the lacZ α fragment permits blue/white color selection of recombinant clones.

Construction of a Soil DNA Library in pMBD14.

DNA isolated from a Massachusetts soil was used to construct a partial BamHI library in pMBD14. The insert size of a random subset of clones ranged from 11.5 to 110 kb, with an average insert size of 47.5 Kb. This library, referred to as the BamBAC library, contains 13,000 clones. Individual clones from this library with insert sizes ranging from 48 to 110 kb were used to test the size limit for conjugation into S. lividans, comparing two E. coli donor strains: ET12567/pUB307 and DH10B/pUB307. ET12567/pUB307, a DNA methylation-deficient (dam⁻, dcm⁻) strain, is used routinely for efficient DNA transfer into methyl-DNA restricting streptomycetes (7). This strain is 5-10 fold more efficient for transfer into S. lividans, which is largely non-restricting, than a methylation proficient strain. Although DH10B is not DNA methylation-deficient, it can be a more suitable donor since it is known to be particularly efficient for the uptake of large DNA (25). Thus it may well reduce any possible bias against large clones in the BamBAC library. Results of the conjugation experiments, shown in Table 3, show that both donor strains were able to transfer BamBAC clones as large as 110 Kb, significantly larger than any such conjugation previously reported in the literature. These data demonstrate that pMBD14 is a significant improvement over existing shuttle vectors for the transfer of large DNA fragments from E. coli into S. lividans.

TABLE 3
Insert size limit for conjugation

| Soil BamBAC clones | DH10B/pUB307 | ET 12567/pUB307 |
|---|---|---|
| 45 Kb | +^{a} | + |
| 65 Kb | − | + |
| 80 Kb | − | + |
| 110 Kb | + | + |

^{a}“+” and “−” indicate successful or unsuccessful conjugation into S. lividans, respectively.

High Throughput Transfer of Environmental DNA Libraries into S. lividans.

We have developed a high throughput method for the transfer of large insert libraries constructed in pMBD14 (described in detail in the Material and Methods section). Using this method, up to 75 clones from a 96-well plate (78%) can be successfully conjugated using either DH10B/pUB307 or ET12567/pUB307 as donors. This novel and heretofore-unknown method is highly efficient and allows for the rapid transfer of large insert DNA libraries into S. lividans in a semi-automated fashion.

### Discussion

Set forth herein is a new and useful vector, pSrpsL, for gene replacement in Streptomyces spp. that can be used to introduce genetically unmarked mutations into the chromosome. This vector provides a significant improvement over heretofore known vectors, such as pGM160 (Hoechst AG) and pRHB514 (described in reference (8)), because it comprises a counterselectable marker for Streptomyces (rpsL) that allows positive selection of rare genetic events that lead to loss of plasmid sequences, and a selectable marker for Streptomyces (tsr, which confers Thio resistance). Using a plasmid and the method set forth herein (FIG. 2), tsr is used to select transformants in Streptomyces, but is subsequently lost after the second crossover event, leaving no drug resistance marker behind in the chromosome. This is vital in many applications, such as the definition of structure-function relationships or the production of vaccine candidates (17). Furthermore, the presence of the selection marker in the plasmid can allow for the recovery of the excised molecule, and thus it can be used to isolate the replaced allele.

Also set forth herein are novel S. lividans strains containing complete and unmarked deletions of either one or both pigmented antibiotic gene clusters (act and red clusters). Other Streptomyces strains have been engineered to lack actinorhodin and undecylprodigiosin, e.g. CH999 (U.S. Pat. No. 5,672,491, (14) an S. coelicolor strain containing a deletion of the act cluster (Δact::ermE) in a red minus background (redE60)), K4-114 and K4-115 (24) (S. lividans TK24 derivatives containing the same deletion of the act cluster (Δact::ermE), in a wild-type red background), and another TK24 derivative with a deletion of the act cluster (Δact::spec) (set forth in U.S. Pat. No. 6,057,103). A strain of the present invention however differs from such heretofore known strains because no antibiotic resistance markers have been introduced that could interfere with future genetic screens, and the red mutation is a deletion of the entire gene cluster, rather than a point mutation. Hence, a strain of the present invention offers a cleaner background for the assessment of novel heterologous activities in that it contains no residual undecylprodigiosin, actinorhodin, or intermediates from their biosynthetic pathways, nor it is possible for the strain to revert spontaneously to a producing phenotype.

Also provided herein are portable DNA cassettes that provide the necessary functions for conjugative transfer of a plasmid from E. coli into Streptomyces spp., and subsequent integration of that plasmid into the Streptomyces chromosome by site-specific recombination. These cassettes of the present invention can be easily transferred in vitro by cre-lox recombination to any existing plasmid containing a loxP site, including BAC vectors. Thus, they allow existing E. coli plasmids to be converted to E. coli-Streptomyces shuttle plasmids using only commercially available products, without the performance of cumbersome and laborious cloning procedures. A cassette of the present invention can be used to create new vectors for library construction, or to modify existing plasmids or libraries. pMBD-1, a plasmid containing a cassette that uses pSAM-2-mediated recombination, is set forth in published PCT Patent Application WO 01/40497. A plasmid of the present invention differs from pMBD-1 in that it (i) uses the ΦC31 integrase system, which has been reported in the literature to be more specific or stable in certain Streptomyces species (10), and (ii) has been modified to remove restriction sites that would restrict the use of its derivatives for library construction. It is shown here that efficient conjugation occurs using a vector of the present invention, while conjugation using pMBD-1 is unsuccessful using the methods described here.

Also provided herein are two shuttle bacterial artificial chromosome (BAC) vectors containing the cassettes described in (3) (pMBD13 and pMBD14) which can be used to construct large insert DNA libraries that can be transferred from E. coli to Streptomyces spp. Other BAC vectors that can be transferred to Streptomyces include:

pPAC-S1 and pPAC-S2 (23), which use the ΦC31 integration system.

a freely-replicating BAC containing the pSCP2* ori (U.S. Pat. No. 6,242,211).

One serious limitation of these latter BACs is that they need to be transferred to Streptomyces by PEG-mediated protoplast transformation, a method that is not amenable for high-through-put screening. In contrast, vectors of the present invention are clearly an improvement because they can be easily transferred to Streptomyces by conjugation.

Other BAC vectors designed to conjugate into Streptomyces include the integrating pMBD-5 and pMBD-6 (pSAM2-derived, WO 01/40497), pMBD-3 (ΦC31-based, WO 01/40497). Our vectors differ from the above in that they allow reproducible, high efficiency transfer into Streptomyces. In addition, pMBD14 has been successfully used to construct a large insert-size soil DNA library, and to efficiently transfer clones (with up to 110 Kb inserts) of such library from E. coli to Streptomyces. It is believed that no other example is available of conjugation of plasmids with such high molecular weight between E. coli and Streptomyces.

A High Throughput Method to Transfer DNA Libraries into Streptomyces spp.

The simplicity and high efficiency of our method makes feasible the transfer of entire large-insert DNA libraries into Streptomyces. Libraries are transferred on a one-to-one clone basis, and thus the E. coli counterpart of any interesting Streptomyces clone is readily available for isolation of DNA for sequencing and/or genetic manipulation. In addition, once the E. coli library is arrayed, it can be transferred to different Streptomyces strains with little additional effort. Those could include other S. lividans strains or different species, such as S. coelicolor and S. ambofaeciens.

Genetic Engeneering of Pseudomonas putida to Accept Shuttle BAC Vectors.

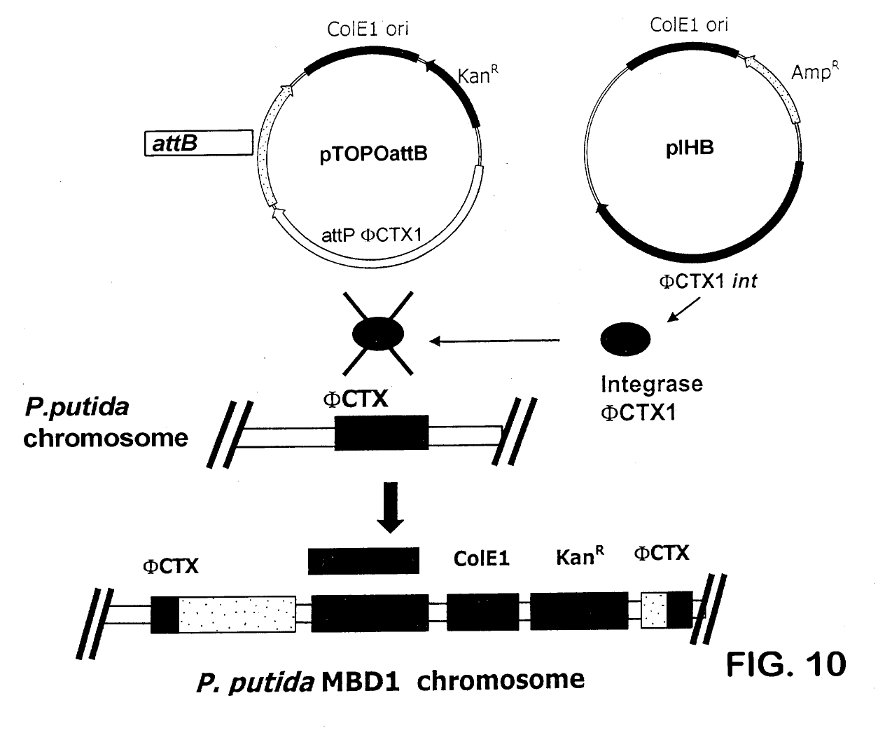

In an effort to continue expanding the host repertoire for our environmental libraries, we have developed Pseudomonas spp. host. We have chosen a non-pathogenic soil Pseudomonas species, P. putida, as candidate for new host development. The first step is to introduce the ΦC31 (Streptomyces phage) attB site in the chromosome of P. putida to allow the use of our E. coli-Streptomyces shuttle BAC vectors without further alteration. This process is summarized in FIG. 10. We used the site-specific integration system of P. aeruginosa ΦCTX phage (30) to deliver the ΦC31 attB site to the P. putida chromosome. First, we cloned the ΦCTX (P. aeruginosa phage) attP site from plasmid p1000 into pTOPOattB, a kanR pUC derivative containing the ΦC31 attB site of S. lividans. The resulting plasmid, p2.10, was cotransformed with pIHB (a pUC plasmid encoding the ΦCTX integrase (30) into P. putida KT-2440. Both plasmids are suicide vectors incapable of replicating in Pseudomonas, but the ΦCTX integrase in pIHB can work in trans to catalyze integration of ΦCTX attP-containig plasmids into the corresponding attB site in the pseudomonas chromosome. Thus, by plating in kanamycin plates after transformation we can select for integration of p2.10 in the ΦCTX attB site, which places the ΦC31 attB site in the pseudomonas chromosome, ready to receive our shuttle BAC vectors. We were able to obtain KanR clones in P. putida KT2440. The presence of the ΦC31 attB site in the pseudomonas chromosome was veryfied by Southern hybridization. The resulting P. putida strain was named MBD1.

The E. coli-Streptomyces Shuttle BAC Vectors can be Introduced and Mantained in P. putida MBD1.

Next, we tested if our library vector, pMBD14, could be introduced into the new P. putida MBD1 strain containing the ΦC31 attB. The RK2 system (which we use to introduce pMBD14 and derivatives into Streptomyces) is routinely used to transfer plasmids from E. coli to Pseudomonads by conjugation. Therefore, we use standard protocols to conjugate DH10B pUB307 containing pMBD14 with our new P. putida MBD1 strain. We were succsessful in obtaining apramycin resistant colonies after conjugation, indicating that the ΦC31 integrase gene and the apramycin resistance gene in pMBD14 were expressed and functional in P. putida.

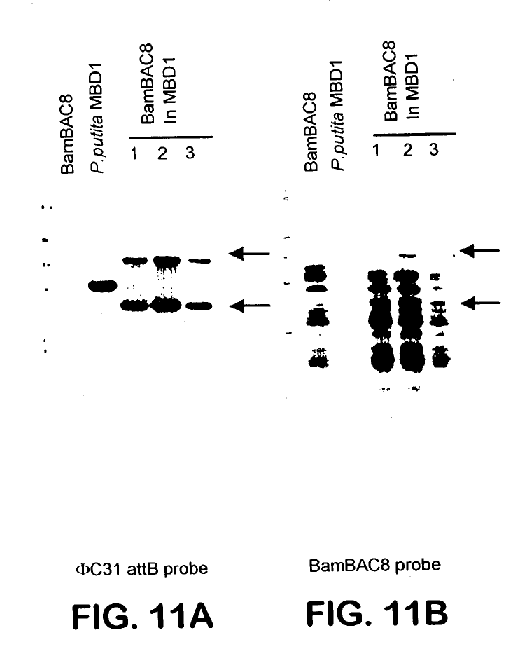

We have also been able to conjugate shuttle BAC derivatives containing DNA inserts of various sizes. These include pGran (37 kb), pSMG1.1 (27 kb), and pSDAPG (6.5 kb)(described below), as well as clones from a soil DNA library constructed in pMBD14. Exconjugants containing one of the soil DNA clones, BamBAC8 (40 kb insert), were analyzed by Southern hybridization to demonstrate that the BAC vector integrated at the ΦC31 site in the P. putida MBD1 chromosome, and suffered no major deletions or rearrangements. The results (FIG. 11) show that, as expected, the band containing the ΦC31 attB site in MBD1 (lane 2) is replaced in the exconjugants (lanes 3-5) by two new bands containing the attL and attR sites created by integration of BamBAC8 in the chromosome. The same two new bands also hybridize to the BamBAC8 probe, showing that they indeed contain the chromosome/integrated vector junctions. Other than the new junction bands, the band pattern after hybridization to the BamBAC8 probe in the exconjugants, is identical to that of the purified BamBAC8, demonstrating that no major deletions or rearrangements of the plasmid have occurred in the 40 kb insert.

These results demonstrate that the E. coli-Streptomyces shuttle BAC vectors can be introduced and mantained in P. putida MBD1. This is the first described example of BAC vectors that can be shuttled between E. coli, Streptomyces and Pseudomonas.

Expression of Heterologous Gene Clusters Encoded by Shuttle BAC Vectors in P. putida MBD 1.

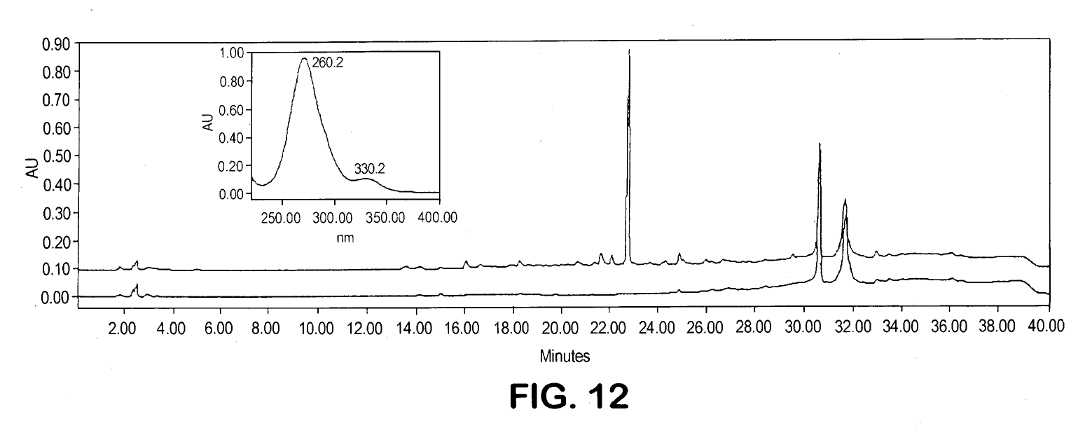

One application of the new P. putida MBD1 strain and shuttle BAC vector system is to allow expression of heterologous small molecules of potential commercial interest in Pseudomonas. As prove of concept, we introduced in P. putida MBD1 a series of BAC constructs containing gene clusters for the synthesis of known antibiotics. The constructs tested are pSgran, pSMG1.1, and pSDAPG, which are shuttle BACs with the granaticin cluster of S. violaceoruber, the MG1.1 soil DNA fragment, and the 2,4-diacetylphloroglucinol cluster of P. fluorescens Q2-87, respectively. P. putida MBD1 exconjugants containing pSMg1.1, pSGran, pDAPG, and the pMBD14 control were grown in 50 ml of YM medium for 6 days at 27° C. Ethyl acetate extracts were prepared and analyzed as described in the Metohds section. 2,4-diacetylphloroglucinol was clearly detectable in the extracts of the P. putida MBD1 clone containing the pSDAPG construct (FIG. 12), demonstrating that the strain of the present invention can be used as surrogate host for the expression of heterologous small molecules.

In addition, 2,4-diacetylphloroglucinol could not be detected in extracts of E. coli DH10B or S. lividans Δact/Δred containing the pSDAPG construct underscoring the unique properties of P. putida MBD1 as an expression host. Conversely, the products of the MG1.1 and granaticin gene clusters, expressed in E. coli and S. lividans respectively, could not be detected in P. putida. This results clearly demonstrate the advantages of the shuttle BAC vectors and multiple host system described here, in that they allow to transfer of genes among a diverse set of bacterial hosts thus increasing the chances of succeeding in obtaining detectable expression of the molecules of interest.

High Through-Put Inergenic Conjugation Method for the Transfer of Shuttle BAC Plasmids into P. putida MBD1 and Analyses of Extracts for Production of Small Molecules.

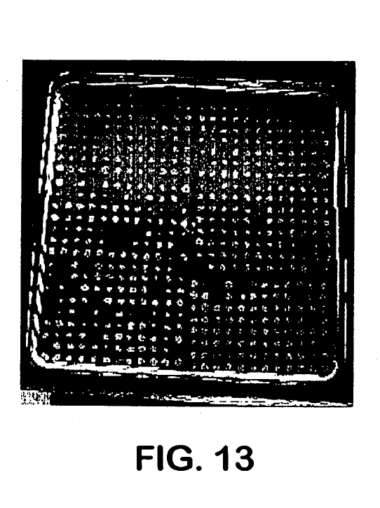

We have developed a HTP conjugation method for P. putida that can be performed in parallel to the E.coli-Streptomyces conjugations. Briefly, a DNA library constructed in pMBD14 is transformed into the E. coli donor strain, DH10B pUB307. Individual clones are picked by a Q-bot into deep 96-well plates containing 2 ml of LB with the appropriate drugs. When they have reached stationary phase, donor cultures are diluted 1:10 into fresh LB without drugs. The same donor dilutions are used for conjugations into S. lividans and P. putida. For P. putida, a 96-pin replicator is used to deliver an aliquot of the donor cultures into 96-well plates containing 50 μl of a P. putida MBD1 exponential culture that has been incubated at 42° C. for 15 min to inactivate restriction systems. The same replicator is used to deliver aliquots of the mixes into an LB Q-bot plate. The plate is incubated overnight at 30° C. P. putida exconjugants containing our library clones are selected by replicating the colonies in the LB plate into an M9 benzoate plate with Apra30 and Nal20. Only exconjugants can grow in this medium. Colonies are visible after 2-3 days of incubation (FIG. 13). The success rate is above 90%.

In addition, we have also developed a HTP extract preparation and analysis methods modeled after our Streptomyces protocols. We grew the host P. putida MBD1, and exconjugants containing the granaticin cluster and MG1.1 in 150 μl of different media in shallow 96-well plates for 4 days. We included P. fluorescens ATCC49323, producer mupiromicin (a polyketide antibiotic), as positive control. We prepared crude methanol extracts using the method previously developed for Streptomyces, and tested the extracts for antibacterial and antifungal activity using Bacillus subtilis and Candida albicans as tester strains, respectively. We found that our host, P. putida MBD1, produces no detectable antibacterial or antifungal compounds under conditions that allow detection of mupiromicin from the positive control. Therefore, P. putida MBD1 provides a “clean” background for production and detection of new antifungals and antibacterials. Finally, no antibacterial activity was detected in the granaticin and MG1.1 exconjugants. These results indicate that environmental libraries built using our shuttle BAC vectors, pMBD14 and pMBD13, can also be screened in P. putida without further modification using the methods described here.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

### References

1. Bechthold, A., J. K. Sohng, T. M. Smith, X. Chu, and H. G. Floss. 1995. Identification of Streptomyces violaceoruber Tu22 genes involved in the biosynthesis of granaticin. Mol Gen Genet 248(5):610-20.

2. Bierman, M., R. Logan, K. O'Brien, E. T. Seno, R. N. Rao, and B. E. Schoner. 1992. Plasmid cloning vectors for the conjugal transfer of DNA from Escherichia coli to Streptomyces spp. Gene 116(1):43-9.

3. Brady, S. F., C. J. Chao, J. Handelsman, and J. Clardy. 2001. Cloning and Heterologous Expression of a Natural Product Biosynthetic Gene Cluster from eDNA. Org Lett 3(13):1981-4.

4. Brady, S. F., and J. Clardy. 2000. Long-chain N-acyl amino acid antibiotics isolated from heterolugously expressed environmental DNA. J. Am. Chem. Soc. 122:12903-12904.

5. Coco, E. A., K. E. Narva, and J. S. Feitelson. 1991. New classes of Streptomyces coelicolor A3(2) mutants blocked in undecylprodigiosin (Red) biosynthesis. Mol Gen Genet 227(1):28-32.

6. Fernandez-Moreno, M. A., E. Martinez, J. L. Caballero, K. Ichinose, D. A. Hopwood, and F. Malpartida. 1994. DNA sequence and functions of the actVI region of the actinorhodin biosynthetic gene cluster of Streptomyces coelicolor A3(2). J Biol Chem 269(40):24854-63.

7. Flett, F., V. Mersinias, and C. P. Smith. 1997. High efficiency intergeneric conjugal transfer of plasmid DNA from Escherichia coli to methyl DNA-restricting streptomycetes. FEMS Microbiol Lett 155(2):223-9.

8. Hosted, T. J., and R. H. Baltz. 1997. Use of rpsL for dominance selection and gene replacement in Streptomyces roseosporus. Journal of Bacteriology 179:180-186.

9. Kieser, T., M. J. Bibb, M. J. Buttner, K. F. Chater, and D. A. Hopwood. 2000. Practical Streptomyces genetics. The John Innes Foundation, Norwich, England.

10. Kuhstoss, S., M. A. Richardson, and R. N. Rao. 1991. Plasmid cloning vectors that integrate site-specifically in Streptomyces spp. Gene 97(1):143-6.

11. MacNeil, I. A., C. L. Tiong, C. Minor, P. R. August, T. H. Grossman, K. A. Loiacono, B. A. Lynch, T. Phillips, S. Narula, R. Sundaramoorthi, A. Tyler, T. Aldredge, H. Long, M. Gilman, D. Holt, and M. S. Osburne. 2001. Expression and isolation of antimicrobial small molecules from soil DNA libraries. J Mol Microbiol Biotechnol 3(2):301-8.

12. Malpartida, F., J. Niemi, R. Navarrete, and D. A. Hopwood. 1990. Cloning and expression in a heterologous host of the complete set of genes for biosynthesis of the Streptomyces coelicolor antibiotic undecylprodigiosin. Gene 93(1):91-9.

13. Martinez-Costa, O. H., A. J. Martin-Triana, E. Martinez, M. A. Fernandez-Moreno, and F. Malpartida. 1999. An additional regulatory gene for actinorhodin production in Streptomyces lividans involves a LysR-type transcriptional regulator. J Bacteriol 181(14):4353-64.

14. McDaniel, R., S. Ebert-Khosla, D. A. Hopwood, and C. Khosla. 1993. Engineered biosynthesis of novel polyketides. Science 262(5139):1546-50.

15. Muth, G., B. Nussbaumer, W. Wohlleben, and A. Puhler. 1989. A vector system with temperature-sensitive replication for gene disruption and mutational cloning in streptomycetes. Molecular and General Genetics 219:341-348.

16. Pelicic, V., M. Jackson, J.-M. Reyrat, W. B. Jacobs, Jr, B. Gicquel, and C. Guilhot. 1997. Efficient allelic exchange and transposon mutagenesis in Mycobacterium tuberculosis. Proceedings of the National Academy of Sciences USA 94:10955-10960.

17. Reyrat, J. M., V. Pelicic, B. Gicquel, and R. Rappuoli. 1998. Counterselectable markers: untapped tools for bacterial genetics and pathogenesis. Infection and Immunity 66:4011-4017.

18. Rondon, M. R., P. R. August, A. D. Bettermann, S. F. Brady, T. H. Grossman, M. R. Liles, K. A. Loiacono, B. A. Lynch, I. A. MacNeil, C. Minor, C. L. Tiong, M. Gilman, M. S. Osburne, J. Clardy, J. Handelsman, and R. M. Goodman. 2000. Cloning the soil metagenome: a strategy for accessing the genetic and functional diversity of uncultured microorganisms. Appl Environ Microbiol 66(6):2541-7.

19. Sambrook, J., E. F. Fritsch, and T. Maniatis. 1989. Molecular cloning: a laboratory manual, 2nd edition. Cold Spring Harbor Laboratory, Cold Spring Harbor, N.Y.

20. Sauer, B. 1994. Site-specific recombination: developments and applications. Curr Opin Biotechnol 5(5):521-7.

21. Shima, J., A. Hesketh, S. Okamoto, S. Kawamoto, and K. Ochi. 1996. Induction of actinorhodin production by rpsL (encoding ribosomal protein S12) mutations that confer streptomycin resistance in Streptomyces lividans and Streptomyces coelicolor A3(2). Journal of Bacteriology 179:7276-7284.

22. Shizuya, H., B. Birren, U. J. Kim, V. Mancino, T. Slepak, Y. Tachiiri, and M. Simon. 1992. Cloning and stable maintenance of 300-kilobase-pair fragments of human DNA in Escherichia coli using an F-factor-based vector. Proc Natl Acad Sci USA 89(18):8794-7.

23. Sosio, M., F. Giusino, C. Cappellano, E. Bossi, A. M. Puglia, and S. Donadio. 2000. Artificial chromosomes for antibiotic-producing actinomycetes. Nat Biotechnol 18(3):343-5.

24. Ziermann, R., and M. C. Betlach. 1999. Recombinant polyketide synthesis in Streptomyces: engineering of improved host strains. Biotechniques 26(1):106-10.

25. Zimmer, R., and A. M. Verrinder Gibbins. 1997. Construction and characterization of a large-fragment chicken bacterial artificial chromosome library. Genomics 42(2):217-26.

27. Wang, Z. et al., 1995. Site-Specific Integration of the Phage Phi CTX Genome into the Pseudomonas Aeruginosa Chromosome: Characterization of the Functional Integrase Gene Located Close to and Upstream of AttP. Mol Gen Genet 246(1) 72-29.

28. Bangera, M G et al. 1996. Characterization of a Genomic Locus Required for Synthesis of the Antibiotic 2,4-Diacetylphloroglucinol by the Biological Control Agent Pseudomonas Fluorescens Q2-87. Mol Plant Microbe Interact 9(2) 83-90.

29. Bonsall, R F et al. 1997. Quantification of 2,4-Diacetylphloroglucinol Produced by Fuorescent Pseudomonas spp. In Vitro and in the Rhizosphere of Wheat. Appl. Environ. Microbiol. 63(3)951-955.

What is claimed is:

1. A vector for introducing genetically unmarked mutations into the chromosome of a unicellular host, comprising:
(a) an origin of replication;
(b) a counterselectable marker for the unicellular host; and
(c) a selectable marker for the unicellular host,
wherein said selectable marker is exciseable by crossover in the unicellular host, so that the selectable marker does not remain in the genome of said unicellular host after crossover.

2. The vector of claim 1, wherein the counterselectable marker is counterselectable in Streptomyces and Pseudomonas species, and the selectable marker is selectable in Streptomyces and Pseudomonas species.

3. The vector of claim 1, wherein said origin of replication comprises the temperature-sensitive origin of replication of plasmid pSG5.

4. The vector of claim 3, wherein said selectable marker confers resistance to thiostrepton.

5. The vector of claim 4, wherein said counterselectable marker comprises rpsl.

6. The vector of claim 5, selected from the group consisting of pGM160rpsL14, having an ATCC accession number of PTA-3850; and pSrpsL6, having an ATCC accession number of PTA-3849.

7. A genetically modified S. lividans strain that is unable to produce actinorhodin, wherein the modification comprises the unmarked deletion of the act gene cluster in the genome of said strain.

8. The genetically modified S. lividans strain of claim 7, having an ATCC accession number of PTA-3847.

9. A genetically modified S. lividans strain that is unable to produce undecylprodigiosin, wherein the modification comprises the deletion of the red gene cluster in the genome of said strain.

10. The genetically modified S. lividans strain of claim 9, having an ATCC accession number of PTA-3848.

11. A genetically modified S. lividans strain that is unable to produce actinorhodin and undecylprodigiosin, wherein the modification comprises the deletion of the act and red gene clusters in the genome of said strain.

12. The genetically modified S. lividans strain of claim 11, having an ATCC accession number of PTA-3846.

13. A genetically modified Pseudomonas strain having a genome containing an attB site.

14. The genetically modified Pseudomonas strain of claim 13, wherein the attB site is the attB site for phage ΦC31.

15. The genetically modified Pseudomonas strain of claim 13, which is a P. Putida strain.

16. The P. putida strain of claim 15, wherein the attB site is the attB site for phage ΦC31.

17. The P. putida strain of claim 16, which is P. putida MBD1, having an ATCC accession number of PTA-3847.

18. A DNA cassette for conjugative transfer of a vector from E. coli into a Streptomyces or Pseudomonas spp., and subsequent integration of said vector into the chromosome of said Streptomyces or Pseudomonas spp., wherein said cassette is inserted into said vector, said cassette comprising:
(a) a first loxP site;
(b) a DNA sequence that encodes a phage integrase operably associated with a promoter;
(c) an attP site for integration of a DNA sequence contained in the vector into the chromosome of the Pseudomonas or Streptomyces spp.;
(d) an origin of transfer for conjugation; and
(e) a second loxP site,
such that elements (b), (c), and (d) lay between elements (a) and (e) in the DNA cassette.

19. The cassette of claim 18, where said integrase and attP site are from phage ΦDC31.

20. The cassette of claim 18, further comprising an antibiotic resistance gene.

21. A vector comprising a DNA cassette for the conjugal transfer of said vector from E. coli into Streptomyces where said cassette comprises:
(a) a first loxP site;
(b) a gene that encodes for integrase operably associated with a promoter;
(c) an attP site for integration of said vector into the chromosome of Streptomyces spp.;
(d) an origin of transfer for conjugation; and
(e) a second loxP site,
wherein elments (b), (c), and (d) lay between elments (a) and (e) in the DNA cassette; and
(f) a counterselectable marker sacB,
such that said vector can be used as a donor for in vitro transfer of the said cassette to a new vector by site-specific recombination.

22. The vector of claim 21, selected from the group consisting of pMBD7, pMBD9, and pMBD12.

23. A bacterial artificial chromosome that can be used to clone large DNA fragments and transfer them from E. coli to Pseudomonas or Streptomyces by conjugation, wherein said bacterial artificial chromosome comprises:
(a) a first loxP site;
(b) a gene that encodes for integrase operably associated with a promoter
(c) an attP site for integration of said vector into the chromosome of Pseudomonas or Streptomyces spp.;
(d) an origin of transfer for conjugation; and
(e) a second loxP site,
wherein elements (b), (c) and (d) lay between elements (a) and (e) in the bacterial artificial chromosome.

24. The bacterial artificial chromosome of claim 23, further comprising an antibiotic resistance gene.

25. The bacterial artificial chromosome of claim 23, further comprising a unique restriction site such that said large DNA fragment is cloned into said bacterial artificial chromosome via said unique restriction site.

26. The bacterial artificial chromosome of claim 25, which is pMBD14, SEQ ID NO: 19.

27. The bacterial artificial chromosome of claim 23, further comprising BSTXI sites such that said large DNA fragment is cloned into said bacterial artificial chromosome via said BSTXI sites, and a high copy origin of replication is removed from said bacterial artificial chromosome.

28. The bacterial artificial chromosome of claim 27, which is pMBD13.

29. A method for the high-throughput transfer of large DNA fragments from E. coli to Streptomyces or Pseudomonas, comprising the steps of:
(a) providing an E. coli cell having a recombinant bacterial artificial chromosome (BAC) containing a large DNA fragment;
(b) providing a Streptomyces or Pseudomonas cell into which the large DNA fragment is to be transferred;
(c) contacting the E. coli cell and the Streptomyces or Pseudomonas cell so that they undergo conjugation, in which the recombinant BAC is transferred to the Streptomyces or Pseudomonas cell; and
(d) selecting the Streptomyces or Pseudomonas cell that contains the recombinant BAC.

30. The method of claim 29, wherein the recombinant BAC integrates into the Streptomyces or Pseudomonas chromosome.


SEQUENCE LISTING (19 sequences)

SEQ ID NO: 1 - DNA, 26 bp, Artificial Sequence
  PCR primer
ggaattccttcgtccgccacgacacg

SEQ ID NO: 2 - DNA, 26 bp, Artificial Sequence
  PCR primer
ggaattccgtcttgcccgcgtcgatg

SEQ ID NO: 3 - DNA, 30 bp, Artificial Sequence
  PCR primer
gaagatcttcggcagcgcgtcagggtgtca

SEQ ID NO: 4 - DNA, 32 bp, Artificial Sequence
  PCR primer
ggaattcctactgcctggtgctcaccgtccac

SEQ ID NO: 5 - DNA, 36 bp, Artificial Sequence
  PCR primer
ggaattccacgagggtggttggcgtcggaacaaggc

SEQ ID NO: 6 - DNA, 36 bp, Artificial Sequence
  PCR primer
cgggatcccaggaagcacaggacgccgaggacgaac

SEQ ID NO: 7 - DNA, 46 bp, Artificial Sequence
  PCR primer
gacggccaagcttcctcgaccttgtggacctcgtcggtgcgcatca

SEQ ID NO: 8 - DNA, 52 bp, Artificial Sequence
  PCR primer
gatcatcgggtcgtctgtttaaacggtcgtcaggcgctgagcaggctggtgt

SEQ ID NO: 9 - DNA, 48 bp, Artificial Sequence
  PCR primer
cgcctgacgaccgtttaaacagacgacccgatgatccccaaccagtgg

SEQ ID NO: 10 - DNA, 40 bp, Artificial Sequence
  PCR primer
cgggatcccgcggggtcagtacacgtaggggacgaacttc

SEQ ID NO: 11 - DNA, 30 bp, Artificial Sequence
  PCR primer
gtgggtacccgtgggtacctgtgctgcttt

SEQ ID NO: 12 - DNA, 30 bp, Artificial Sequence
  PCR primer
ttgttgaccagtacgtccaccctgccgtgc

SEQ ID NO: 13 - DNA, 30 bp, Artificial Sequence
  PCR primer
agatgcagaagctggacggccgtgacttcg

SEQ ID NO: 14 - DNA, 30 bp, Artificial Sequence
  PCR primer
ggccctggaggatctcatcagcgcgatgtt

SEQ ID NO: 15 - DNA, 29 bp, Artificial Sequence
  PCR primer
tagagggcggacatcccgacgatggcgat

SEQ ID NO: 16 - DNA, 30 bp, Artificial Sequence
  PCR primer
agccgtggtacgggcattcgatggtgttgc

SEQ ID NO: 17 - DNA, 21 bp, Artificial Sequence
  PCR primer
accatcgtgatcggcgtgtgc

SEQ ID NO: 18 - DNA, 24 bp, Artificial Sequence
  PCR primer
gcccgtgatcccgatgttcaccgg

SEQ ID NO: 19 - DNA, 14468 bp, Artificial Sequence
  pMBD14
gggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgatccccggggacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttctgaacggtctggttataggtacattgagcaactgactgaaatgcctcaaaatgttctttacgatgccattgggatatatcaacggtggtatatccagtgatttttttctccattttagcttccttagctcctgaaagatccataacttcgtatagcatacattatacgaagttatattcgatgcggccgcaaggggttcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattcgagctcggtacccggggatcctctagagtcgacctgcaggcatgcaagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgaaccccttgcggccgcccgggccgtcgaccaattctcatgtttgacagcttatcatcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcgcgataagctcatggagcggcgtaaccgtcgcacaggaaggacagagaaagcgcggatctgggaagtgacggacagaacggtcaggacctggattggggaggcggttgccgccgctgctgctgacggtgtgacgttctctgttccggtcacaccacatacgttccgccattcctatgcgatgcacatgctgtatgccggtataccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagcgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatcttcatattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatcagtcgacggtaccggacatatgcccgggaattcctgcaggatcgatccgctcgagaagctttctagtggtaccaacccaggcaggtagcacagtgcgcgcccactgccttacggatagcacgacgcatcttcgccgcagcgttgttcccacgggcgatagcagcacgccgcttcgcatggctcttcacactgcgctgtgcctgataggtggcgtggtgctgagcacagcgcccaccatgagtagcccagtccctacagtcgagacaccgggtacgcatagggcaccccccccccccttccggggacacccccctatccgtcgacacccccctacctactcacgtgagtaggctgggcgcggcagcgctagaagcgaccgcccgtcatgaagtgggcagtgagccaacccatgaaggccagtagcgcaaagcgtcgagcacgcacccaacccgaaggcttgcctgtactgccgctttgcgtggcgaaccatgaccagacgtgttcgctcagcgtgtcgcccggcttcttattgaagagcgctcgaccctcaatgacgccgaacgcggcaagccatgcgagccaagcgattgtgtatccgctcattgagccccccgacggattcgaaccgccgaccccctgattacgaaacaggcgctctaaccgaactgagctagggaggcaacgccggtcgctcaaccctgggagagatcaggggagtgaccggcctgagtgggtagcgctcgacgccgttccttagcggtgcttgatgcgtggcgcgtaaacccctctacctatatgaaggttgtcggcaacctgattcggggtcacagcgaagggcgcccgggtgacgctgtgatgaagtggcgtgaagtctggattacccaatgagaagtcattaggaatgtgtgtttcgagtcattccgtcacagcatcacttcccccaggtcaacggctcgccccgctgtgctgtggtgacggagtcactgttacttaggctaactaactaaaccgagacaagagaaatgggccgaccgggggaccggccgacccttcccgctgccccaggaagcccgctgtgcgcccgtctcagcgcctaacaggcttcccgggtgtctcgctacgccgctacgtcttccgtgccgtcctgggcgtcgtcttcgtcgtcgtcggtcggcggcttcgcccacgtgatcgaagcgcgcttctcgatgggcgttccctgccccctgcccgtagtcgacttcgtgacaacgatcttgtctacgaagagcccgacgaacacgcgcttgtcgtctactgacgcgcgcccccaccacgacttagggccggtcgggtcagcgtcggcgtcttcggggaaccattggtcaaggggaagcttcggggcttcggcggcttcaagttcggcaagccgctcttccgccccttgctgccggagcgtcagcgctgcctgttgcttccggaagtgcttcctgccaacgggtccgtcgtacgcgcctgccgcgcggtcttcgtacagctcttcaagggcgttcagggcgtcggcgcgctccgcaacaaggttcgcccgttcgccgctcttctcaggcgcctcagtgagcttgccgaagcgtcgggcggcttcccacagaagcgccaacgtctcttcgtcgccttcggcgtgcctgatcttgttgaagatgcgttccgcaacgaacttgtcgagtgccgccatgctgacgttgcacgtgccttcgtgctgcccaggtgcggacgggtcgaccaccttccggcgacggcagcggtaagagtccttgatcgattcttccccgcgcttcgaagtcatgacggcgccacactcgcagtacagcttgtccatggcggacagaatggcttgcccccgggaaagccccttgccgcgccccctgccgtccaaccacgcctgaagctcataccactcagcgggctcgatgatcggtccgcaatcaagctcgaccggccggagcgtgatcgggtcgcgctgaatgcggtaaccctcaatcttcgtggtcggcgtgccgtccggcttcttcttgtagatcacctcagcggcgaagcccgcaatacgcgggtcccgaaggattcgcataacggttgccgggtcccaggcgcttgaagcggtcttcttcccaatcgtctcgccccgggtcggcacggcgtcagcgtccatgcgcttacaaagccccgtgatgctgcccgggtgaatggcggcttgactgcccggcttgaagggaaggtgtttgtgcgtcttgatctcacgccaccaccaccggattacgtcgggctcgaactcgaagggtccggtaaggggagtggtcgagtgcgcaagcttgttgatgacgacattgaccattcggccgttgcgcgtgatctccttcgtctccgaaacaagctcgaagccgtaaggcgccttcccgccgacgtacccgcccaattcgcgctgaaggttcttcgtgtcgagaatcttcgccgacttcagcgaagattctttgtgcgacgcgtcgagccgcataatcaggtgaatcaggtccatgacgtttccctgccggaagacgccttcctgagtggaaacaatcgtcacgcccagggcgagcaattccgagacaatcggaatcgcgtccatgaccttcaggcgcgagaagcgcgacacgtcatagacaatgatcatgttgagccgcccggcgcggcattcgttcaggatgcgttcgaactccgggcgctccgccgtcccgaacgccgacgtgcccggcgcttcgctgaaatgcccgacgaacctgaaccggcccccgtcgcgctcgacttcgcgctgaaggtcggccgccttgtcttcgttggcgctacgctgtgtcgctgggcttgctgcgctcgaattctcgcgctcgcgcgactgacggtcgtaagcacccgcgtacgtgtccaccccggtcacaaccccttgtgtcatgtcggcgaccctacgcccccaactgagagaactcaaaggttaccccagttggggcactactcccgaaaaccgcttctgacctgggaaaacgtgaagccccggggcatccgctgagggttgccgccggggcttcggtgtgtccgtcagtacgggccatagaggggcgtcgtgtaacccacccagggcgattgctccggcacggggaagccgcgccacgccttcgggacgtcagcggctcgccccgtgcgctcccacatctgccacgtctgaaggcagtgggcgagccagtcatacgccgccgaccgcgaacgccattcgagcgtcaccggctcgccgtcgtcgtcctggggcagcgcgcagtaatcgcccatgacttcatcgaagatcacgaacggcggattcttgcgcacgacccaggttccgtttttccggcgctgtagcgcgcgttccatgaccctgtatctcttcaccggccgacccgctcacgcgcgacgttgtacgggacggcgcccccgccgacaaggggcacttcgccccacgtccggtagcaccgcattagccacgcctgggcgccatgctgggtgacccattcgagcggcatgagggttgaggggtcgccgtcgtcgggcagcgcgcaatagcccttcatggctccgtcgaagatcacccaggggcgcacgacgggcgggttgccgtcttcggttcgctgtagttcctgccgccactcaggcggacgctgtgtgactcgataccgacgcatagggaacacgctaggggacttccctcagttgggggagagcgtgacgaaaattccaccttcgcgggcttcccaggtgcgcccccgctcgacctcaaaccggccatcggtcacaagcttcatgcgcaacgggccgaccacgttgaagagaatgtgcgcgagcgtcgtcgtgtcttcgtcggcttcggcttcctcgcgcgcccaggtgtcggcgggtgagtggcccgtgtagcacgtcggcgagtagggcacgcccagggcttccgctagttccttgtcgcacacaacgcccgggtcggcggcagtctccggggtgtcgccccaggcttcttcatacgggtactcaggcgagccgccttcagcgatcaagggcgtctcagcgtcggcttcgtggttgtgccccgggaagggcagcatgacgtctggaatcgatgataagctgtcaaacatgagaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgcccccggacgagcgtctgctccgccattcgccgtccgccgtgccaatcggatcagccgtccaaatgcgggattttcgttagtcggaggccaaacggcattgagcgtcagcatatcatcagcgagctgaagaaagacaatccccgatccgctccacgtgttgccccagcaatcagcgcgaccttgcccctccaacgtcatctcgttctccgctcatgagctcagccactggcgagcggcatcgcattcttcgcatcccgcctctggcggatgcaggaagatcaacggatctcggcccagttgacccagggctgtcgccacaatgtcgcgggagcggatcaaccgagcaaaggcatgaccgactggaccttccttctgaaggctcttctccttgagccacctgtccgccaaggcaaagcgctcacagcagtggtcattctcgagataatcgacgcgtaccaacttgccatcctgaagaatggtgcagtgtctcggcaccccatagggaacctttgccatcaactcggcaagatgcagcgtcgtgttggcatcgtgtcccacgccgaggagaagtacctgcccatcgagttcatggacacgggcgaccgggcttgcaggcgagtgaggtggcaggggcaatggatcagagatgatctgctctgcctgtggccccgctgccgcaaaggcaaatggatgggcgctgcgctttacatttggcaggcgccagaatgtgtcagagacaactccaaggtccggtgtaacgggcgacgtggcaggatcgaacggctcgtcgtccagacctgaccacgagggcatgacgagcgtccctcccggacccagcgcagcacgcagggcctcgatcagtccaagtggcccatcttcgaggggccggacgctacggaaggagctgtggaccagcagcacaccgccgggggtaaccccaaggttgagaagctgaccgatgagctcggcttttcgccattcgtattgcacgacattgcactccaccgctgatgacatcagtcgatcatagcacgatcaacggcactgttgcaaatagtcggtggtgataaacttatcatccccttttgctgatggagctgcacatgaacccattcaaaggccggcattttcagcgtgacatcattctgtgggccgtacgctggtactgcaaatacggcatcagttaccgtgagctgcaggtcgacggatcttttccgctgcataaccctgcttcggggtcattatagcgattttttcggtatatccatcctttttcgcacgatatacaggattttgccaaagggttcgtgtagactttccttggtgtatccaacggcgtcagccg